(19) United States
(12) Patent Application Publication
(10) Pub. No.: US 2010/0105687 A1
(43) Pub. Date: Apr. 29, 2010

(54) METHODS FOR TREATING COGNITIVE DISORDERS USING 1-ARYL-1-HYDROXY-2,3-DIAMINO-PROPYL AMINES, 1-HETEROARYL-1-HYDROXY-2,3-DIAMINO-PROPYL AMINES AND RELATED COMPOUNDS

(75) Inventors: John E. Donello, Dana Point, CA (US); Fabien J. Schweighoffer, Val-de-marne (FR); Lauren M. Luhrs, Rancho Santa Margarita, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/530,141

(22) PCT Filed: Feb. 26, 2008
(86) PCT No.: PCT/US2008/054936

§ 371 Date: Jan. 8, 2010

Related U.S. Application Data
(60) Provisional application No. 60/893,203, filed on Mar. 6, 2007.

(51) Int. Cl. A61K 31/5375; A61K 31/40; A61K 31/4025; A61P 25/00

(52) U.S. Cl. 514/238.2; 514/237.8; 514/428; 514/422; 514/343

(57) ABSTRACT

Disclosed herein are methods of treating a patient suffering from a cognitive disorder using compounds of the following formula, Formula (2), wherein the variables have the meaning defined in the specification.

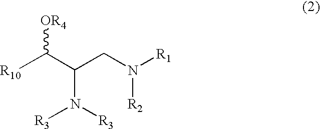

### CROSS REFERENCE TO RELATED APPLICATIONS

This application is based on, and claims the benefit of, U.S. Provisional Application No. 60/893,203, filed Mar. 6, 2007, and which is incorporated herein by reference.

### BACKGROUND OF THE INVENTION

1. Field of the Invention

The present invention is directed to methods of treating a patient suffering from one or more types of cognitive disorders using derivatives of 1-aryl-1-hydroxy-2,3-diamino-propyl amines, 1-heteroaryl-1-hydroxy-2,3-diamino-propyl amines and related compounds.

2. Background Art

1-Phenyl-2-decanoylamino-3-morpholino-1-propanol (PDMP) was discovered by Vunam, R. R. and Radin, N., Chem. Phys. Lipids, 26, 265-278, 1980. Preparation of PDMP is described in Inokuchi, J. et al., J. Lipid Res. 28, 565-571, 1987; Radin, A. et al., NeuroProtocols, 3(2), 145-55, 1993; Abe et al., J. Lipid Res. 36, 611-621, 1995 and U.S. Pat. No. 5,916,911.

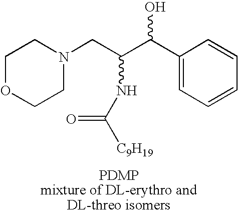

The isomers most active have the R,R-(D-threo)-configuration.

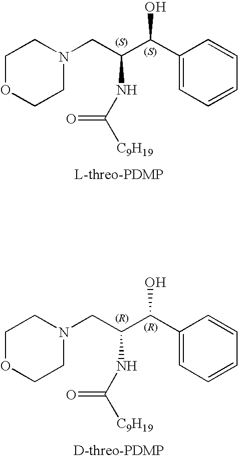

Preparation of enantiomerically pure D-threo-PDMP has been reported by Mitchell, Scott A. [J. Org. Chem., 63 (24), 8837-8842, 1998]; Miura, T. et al., [Bioorg. Med. Chem., 6, 1481-1498, 1998]; Shin, S. et al., [Tetrahedron asymmetry, 11, 3293-3301, 2000]; WO 2002012185

A stereoselective synthesis of enantiomerically pure D-threo-PDMP has also been described by Shin, S. et al., Tetrahedron asymmetry, 11, 3293-3301, 2000 and WO 2002012185 the key step is the regioselective cleavage by nitrogen nucleophiles, as morpholine, of the C(3)-N-bond of non-activated enantiomerically pure aziridine-2-methanols.

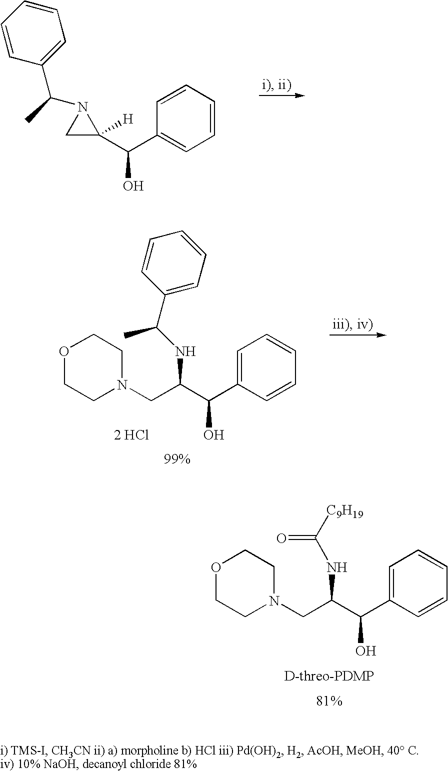

On the other hand, the synthesis of enantiomerically pure (1S,2S)-1-phenyl-2-decanoylamino-3-morpholino-1-propanol (L-threo-PDMP) from L-serine has also been described by Mitchell, Scott A., J. Org. Chem., 63 (24), 8837-8842, 1998.

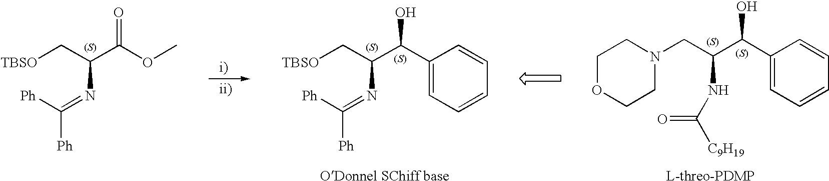

Other known methods to obtain L-threo-PDMP are described by Miura, T. et al, Bioorg. Med. Chem., 6, 1481-1498, 1998 and in JP-A-9-216858.

Synthesis of (1S,2S)-three- and (1R,2S)-erythro-1-phenyl-2-palmitoylamino-3-N-morpholino-1-propanol (PPMP) were described starting from Garner aldehyde of L-serine, by Nishida, A., Synlett, 4, 389-390, 1998.

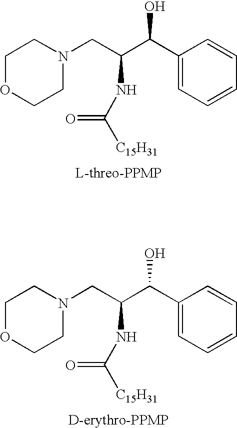

D-threo-1-phenyl-2-palmitoylamino-3-pyrrolidino-1-propanol (P4 or PPPP) analogues were first obtained by a Mannich reaction as described Abe, A. et al., J. Biochem., 111, 191-196, 1992 or U.S. Pat. No. 5,916,911 and WO 2001004108.

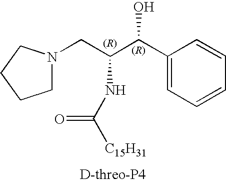

Preparation of D-threo-4′-hydroxy-P4 was described by Lee, L. et al., J. Biol. Chem., 274, 21, 14662-14669, 1999. In addition, a series of dioxane substitutions was designed and tested. These included 3′,4′-methylenedioxyphenyl-3′,4′-ethylenedioxyphenyl-, and 3′,4′-trimethylenedioxyphenyl-substituted homologues.

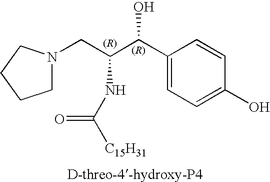

Synthesis of enantiomerically pure D-threo-1-phenyl-2-benzyloxycarbonylamino-3-pyrrolidino-1-propanol (PBPP) and D-threo-P4 and its analogues from N-benzyloxycarbonyl-D-serine, was described by Jimbo M. et al, J. Biochem., 127(3), 485-91, 2000 and EP 782992 (Seikagaku Kogyo Co.).

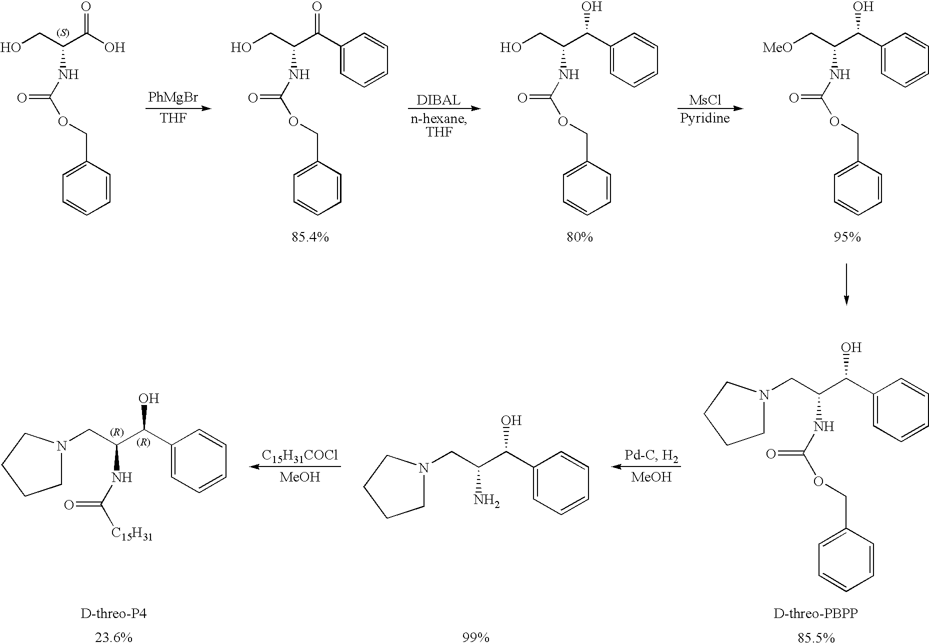

Novel prodrugs of P4 derivatives were described in US 20020198240 and WO 2002062777.

Synthesis of enantiomerically pure of D-threo-ethylenedioxy-P4 and D-threo-p-methoxy-P4 were described by Husain A. and Ganem B., Tetrahedron Lett., 43, 8621-8623, 2002. The key step is a highly syn-selective additions of aryl Grignard reagents to Garner aldehyde.

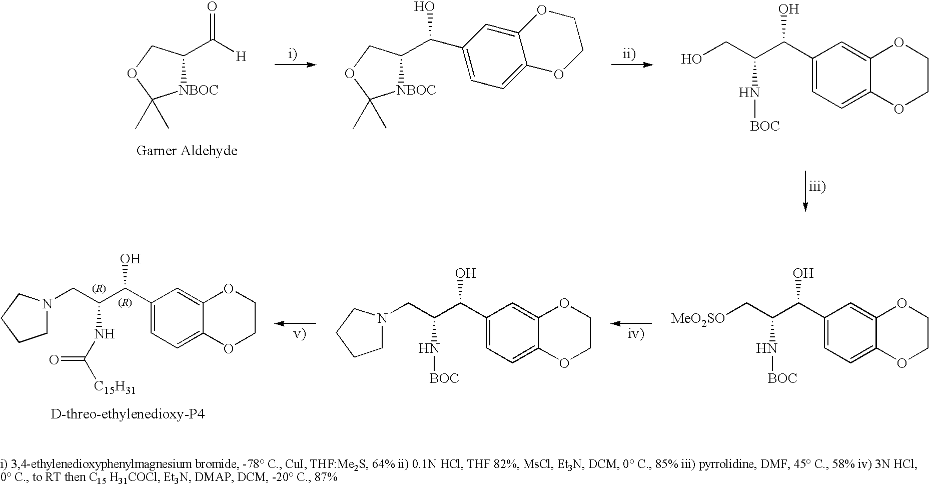

Diastereoselective synthesis of P4 analogues were described in US 03/0153768 and WO 2003045928 (Genzyme Corp.); Oxazolines I [R1=(un)substituted aryl; R², R³=H, (un)substituted aliphatic; NR²R³=heterocyclic] are prepared as intermediates for P4 glucosyltransferase inhibitors from R¹CHO and R²R³NCOCH₂CN. Thus, methyl isocyanoacetate CNCH₂CO₂Me was treated with pyrrolidine and the amide was treated with 1,4-benzodioxane-6-carboxaldehyde, followed by hydrolysis of the oxazoline using HCl in methanol, reduction of the keto group of amide II using LiAlH₄, and acylation with palmitoyl chloride to give D,L-threo-ethylenedioxy-P4 III.

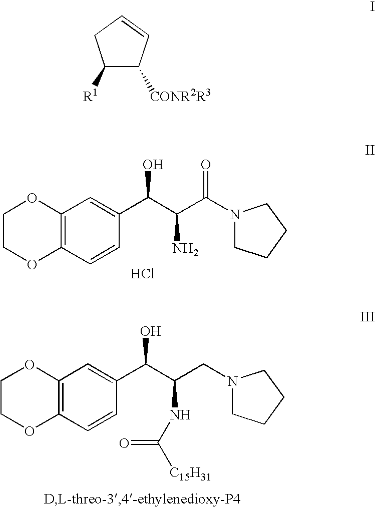

Synthesis of enantiopure P4 analogues were described in WO 2003008399 (Genzyme Corp.).

P4 derivatives, such as I [R¹, R⁵=un(substituted) aromatic; R², R³=H, un(substituted) aliphatic; NR²R³=(un)substituted non-aromatic heterocyclic ring; R⁴=O, H₂], have been prepared. Thus, D-threo-ethylenedioxy-P4 was prepared via a multistep synthetic sequence starting from S-(+)-Ph glycinol, phenyl-α-bromoacetate, 1,4-benzodioxan-6-carboxaldehyde, pyrrolidine and palmitoyl chloride.

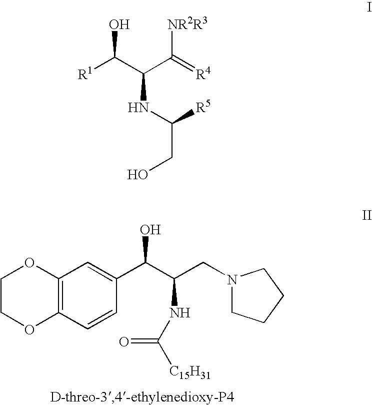

New D-threo-P4 analogues that bear ether substituents on the aromatic ring have been recently synthesized from D-serine and found to suppress neurite extension in an embryonic insect cell line as described by Slavish., J. P. et al., Bioorg. Med. Chem. Lett., 14, 1487-1490, 2004.

Further references which serve as background to the present invention are U.S. Pat. Nos. 5,945,442; 5,952,370; 6,030,995 and 6,051,598; Kurosawa et al, Journal of Labelled Compounds & Radiopharmaceuticals (1996), 38(3), 285-97; Published PCT application WO 01/38228; and Kastron et al. Latvijas PSR Zinatnu Akademijas Vestis, Kimijas Serija (1965) (4), 474-7.

### SUMMARY OF THE INVENTION

The present invention is directed to methods of treating a patient suffering from one or more types of cognitive disorders using compounds of Formula 1:

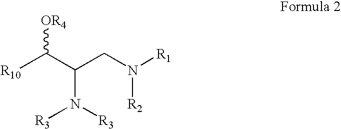

where R₁ is H or alkyl of 1 to 6 carbons,
R₂ is H, alkyl of 1 to 6 carbons or the R₁ and R₂ groups together with the nitrogen form a saturated or unsaturated 4, 5, 6 or 7 membered ring that optionally includes one or two heteroatoms independently selected from N, O and S, said 4, 5, 6 or 7 membered ring optionally being substituted with a halogen, COOH, CH₂OH, OH, B(OH)₂, cyano or with an alkyl group having 1 to 6 alkyl groups;
R₃ is independently selected from H, alkyl of 1 to 20 carbons, aryl or heteroaryl, aryl-alkyl or heteroaryl-alkyl where the alkyl moiety is has 1 to 4 carbons, cycloalkyl of 3 to 6 carbons, said aryl or heteroaryl groups being optionally substituted with 1 to 3 groups independently selected from the group consisting of halogen, alkyl of 1 to 6 carbons, alkoxy of 1 to 6 carbons and thioxy of 1 to 6 carbons, or
R₃ is CO—R₇ or CO—O—R₇ where R₇ is H, alkyl of 1 to 1 to 20 carbons, benzyl, alkyl of 1 to 20 carbons substituted with and NH₂ group, with a NHCOOalkyl or with an NH—COalkyl group where the alkyl group has one to 6 carbons, or R₇ is aryl, heteroaryl, aryl-alkyl or heteroaryl-alkyl where the alkyl moiety is branched or unbranched and has 1 to 4 carbons, said aryl or heteroaryl groups being optionally substituted with 1 to 3 groups independently selected from the group consisting of halogen, alkyl of 1 to 6 carbons, alkoxy of 1 to 6 carbons and thioxy of 1 to 6 carbons;
R₄ is H, alkyl of 1 to 6 carbons or CO—R₈ where R₈ is alkyl of 1 to 6 carbons;
the wavy lines represent bonds connected to carbons having R or S configuration, and
R₁₀ is selected from the groups of formulas (i) and (ii)

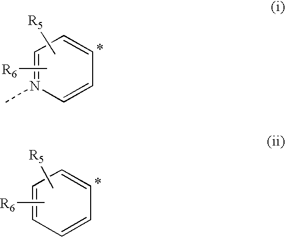

where the * indicates the carbon atom to which the remaining moiety of the molecule is attached;
R₅ and R₆ independently are H, alkyl of 1 to 6 carbons, halogen, alkoxy of 1 to 6 carbons or the R₅ and R₆ groups together with the atoms to which they are attached jointly form a carbocyclic or a heterocyclic ring, the carbocyclic ring having 5 or 6 atoms in the ring, the heterocyclic ring having 5 or 6 atoms in the ring and 1 to 3 heteroatoms independently selected from N, O and S, and said carbocyclic or heterocyclic ring jointly formed by R₅ and R₆ being optionally substituted with 1 to 6 R₉ groups where R₉ is independently selected from halogen, alkyl of 1 to 6 carbons, alkoxy of 1 to 6 carbons, with the proviso:
that when R₁₀ has formula (ii) then Formula 1 does not include compounds where R₄ is hydrogen and R₁ and R₂ jointly with the nitrogen form a morpholin or a pyrrolidin ring and where R₅ and R₆ both are H or one of R₅ and R₆ is OCH₃ and the other is H,
and the present invention is also directed to all pharmaceutically acceptable salts of said compounds.

Any of the compounds described here may be used to treat a patient suffering from a cognitive disorder, such as an agnosia, an amnesia, an aphasia, an apraxia, a delirium, a dementia, and a learning disorder.

### DETAILED DESCRIPTION OF THE INVENTION

A general description of the compounds of the invention is provided in the Summary Section of the present application for patent. Most compounds of the invention contain one or more asymmetric centers, such that the compounds may exist in enantiomeric as well as in diastereomeric forms. In fact, most of the compounds of the present invention have two asymmetric carbons adjacent to one another and therefore can exist in erythro or threo form, with each of these two forms having dextrorotatory (D) or levorotary (L) enantiomers. Although the threo form is generally preferred in accordance with the present invention, unless it is specifically noted otherwise, the scope of the present invention includes all enantiomers, diastereomers and diastereomeric and racemic mixtures. In light of the foregoing, it should be clearly understood that the designation “DL” or “(+/−)” or “(±)” in this application includes the pure dextrorotatory enantiomer, the pure levorotatory enantiomer and all racemic mixtures, including mixtures where the two enantiomers are present in equal or in unequal proportions. Moreover, for simplicity sake in many of the structural formulas, such as in the example below, only one of the enantiomers is actually shown but when the designation “DL” (or “(+/−)” or “(±)”) appears it also includes the enantiomeric form (mirror image) of the structure actually shown in the formula.

For Example:

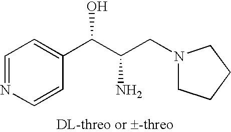

Thus, in the example above, only one enantiomer is shown, but because the designation “DL” (or “(+/−)” or “(±)”) appears below the formula, its optical isomer

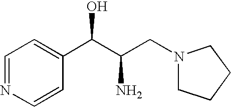

and all racemic mixtures of the two optical isomers are also included.

In the case of some compounds of the present invention one enantiomer of the threo, and in some cases of the erythro, is significantly more active than the other enantiomer of the same pair. Some of the compounds which may be used in the method of the present invention may contain three or more asymmetric centers.

Keeping the foregoing examples in mind a person of ordinary skill in the art should readily understand the scope of each described example, although in a broad sense all isomers, enantiomers and racemic mixtures are within the scope of the invention.

The term “alkyl” in the general description and definition of the compounds includes straight chain as well as branch-chained alkyl groups.

Generally speaking the compounds of the invention may form salts with pharmaceutically acceptable acids or bases, and such pharmaceutically acceptable salts of the compounds of Formula 1 are also within the scope of the invention.

Referring now to the novel compounds of Formula 1, the R₅ and R₆ groups preferably both are independently selected from H, alkyl, alkoxy and still more preferably are H. In the preferred compounds the R₃ groups are preferably both H, or one of the R₃ groups is H and the other is an acyl group or an arylalkylcarbamoyl group. The R₄ group is preferably H (but see the “proviso” in the Summary section) or alkanoyl, and the R₁ and R₂ groups preferably are pyrrolidino or morpholino.

### Biological Activity, Modes of Administration

The compounds described here may be used to treat a patient suffering from one or more types of cognitive disorder, such as an agnosia, an amnesia, an aphasia, an apraxia, a delirium, a dementia, and a learning disorder.

To “treat,” as used here, means to deal with medically. It includes, for example, administering a compound of the invention to prevent the onset of a cognitive disorder, to alleviate its severity, and to prevent its reoccurrence.

The term “cognitive disorder,” as used here, means any condition characterized by a deficit in mental activities associated with thinking, learning, or memory. Examples of such disorders include agnosias, amnesias, aphasias, apraxias, deliriums, dementias, and learning disorders.

In some cases, the cause of a cognitive disorder may be unknown or uncertain. In other cases, the cognitive disorder may be associated with (that is, be caused by or occur in the presence of) other conditions characterized by damage to or loss of neurons or other structures involved in the transmission of signals between neurons. Hence, cognitive disorders may be associated with neurodegenerative diseases such as Alzheimer's disease, corticobasal degeneration, Creutzfeldt-Jacob disease, frontotemporal lobar degeneration, Huntington disease, multiple sclerosis, normal pressure hydrocephalus, organic chronic brain syndrome, Parkinson's disease, Pick disease, progressive supranuclear palsy, or senile dementia (Alzheimer type); it may be associated with truama to the brain, such as that caused by chronic subdural hematoma, concussion, intracerebral hemorrhage, or with other injury to the brain, such as that cause by infection (e.g., encephalitis, meningitis, septicemia) or drug intoxication or abuse.

Cognitive disorders may also be associated with other conditions which impair normal functioning of the central nervous system, including psychiatric disorders such as anxiety disorders, dissociative disorders, mood disorders, schizophrenia, and somatoform and factitious disorders; it may also be associated with conditions of the peripheral nervous system, such as chronic pain.

The compounds described here may be used to treat agnosias, amnesias, aphasias, apraxias, deliriums, dementias, learning disorders and other cognitive disorders regardless of whether their cause is known or not.

Examples of dementias which may be treated with the methods of the invention include AIDS dementia complex, Binswanger's disease, dementia with Lewy Bodies, frontotemporal dementia, multi-infarct dementia, Pick's disease, semantic dementia, senile dementia, and vascular dementia.

Examples of learning disorders which may be treated with the methods of the invention include Asperger's syndrome, attention deficit disorder, attention deficit hyperactivity disorder, autism, childhood disintegrative disorder, and Rett syndrome.

Examples of aphasia which may be treated with the methods of the invention include progressive non-fluent aphasia.

The compounds described here may also be used to treat patient having deficits in mental activities that are mild or that otherwise do not significantly interfere with daily life. Mild cognitive impairment is an example of such a condition: a patient with mild cognitive impairment displays symptoms of dementia (e.g., difficulties with language or memory) but the severity of these symptoms is such that a diagnosis of dementia may not be appropriate. The compounds described here may be used to treat mild cognitive impairment and other, similarly less severe forms of cognitive disorders.

### Examples of Compounds of the Invention

Table 1, below, lists compounds which may be used in the method of the invention.

TABLE 1

| Compound | Chemical Formula |
|---|---|
| 1 | 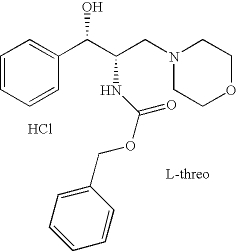 |
| 3 | 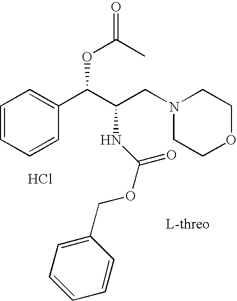 |
| 46 | 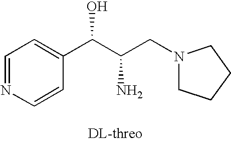 |

### Modes of Administration:

Compounds useful in the methods of the invention may be administered at pharmaceutically effective dosages. Such dosages are normally the minimum dose necessary to achieve the desired therapeutic effect; in the treatment of chromic pain, this amount would be roughly that necessary to reduce the discomfort caused by the pain to tolerable levels. For human adults such doses generally will be in the range of 0.1-5,000 mg/day; more preferably in the range of 1 to 3,000 mg/day, 10 mg to 500 mg/day, 500 to 1,000 mg/day, 1,000 to 1,500 mg/day, 1,500 to 2,000 mg/day, 2,000 to 2,500 mg/day, or 2,500 to 3,000 mg/day. However, the actual amount of the compound to be administered in any given case will be determined by a physician taking into account the relevant circumstances, such as the severity of the pain, the age and weight of the patient, the patient's general physical condition, the cause of the pain, and the route of administration.

The compounds are useful in the treatment of pain in a mammal; particularly a human being. Preferably, the patient will be given the compound orally in any acceptable form, such as a tablet, liquid, capsule, powder and the like. However, other routes may be desirable or necessary, particularly if the patient suffers from nausea. Such other routes may include, without exception, transdermal, intraperitonial, parenteral, subcutaneous, intranasal, intrathecal, intramuscular, intravenous and intrarectal modes of delivery. Compositions useful in the method of the invention may further include an excipient. Such an excipient may be a carrier or a diluent; this is usually mixed with the active compound, or permitted to dilute or enclose the active compound. If a diluent, the carrier may be solid, semi-solid, or liquid material that acts as an excipient or vehicle for the active compound. The formulations may also include wetting agents, emulsifying agents, preserving agents, sweetening agents, and/or flavoring agents. If used as in an ophthalmic or infusion format, the formulation will usually contain one or more salt to influence the osmotic pressure of the formulation.

### Synthetic Methods for Obtaining the Compounds of the Invention, Experimental

The compound of the invention can be synthesized by utilizing the synthetic methods described in the experimental below, or such modifications of the below described experimental methods which will become readily apparent to those skilled in the art in light of the present disclosure.

### General

¹H NMR spectra were recorded at ambient temperature with an Avance 300 (Bruker) spectrometer. The compounds were analyzed by reverse phase high performance liquid chromatography (HPLC) using a Waters Autopurification System equipped with a Waters 2525 Pump, a Waters 2696 photodiode array detector, and a XTerra column (Part. No. 186000482, 5 μm, C18, 4.5×50 mm).

The HPLC method used was a gradient of 5% solvent B to 100% in 7 min. Solvent A was H₂O with 0.05% TFA and solvent B was CH₃CN with 0.05% TFA (Method A).

Melting points were measured with a Büchi B-545 melting point apparatus and were uncorrected. To isolate reaction products the solvent were removed by evaporation using a vacuum rotatory evaporator, the water bath temperature not exceeding 40° C.

### General Synthetic Routes

The compound of the invention can be synthesized by utilizing the synthetic methods described in a general sense immediately below and in more detail in the experimental section of the present application, or by such modifications of the below described general and experimental methods which will become readily apparent to those skilled in the art in light of the present disclosure.

A general synthetic route to the compound of the present invention which are substituted “1-hydroxyl-propyl amines” may lead through the synthesis of the corresponding substituted “3-hydroxyl-propyl amide” compounds, followed by reduction of the carbonyl group of the “carboxylic acid amide” moiety with a reducing agent such as lithium aluminum hydride, or like reducing agent.

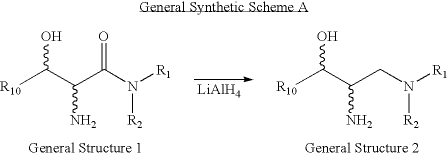

This reaction is illustrated in General Synthetic Scheme A, where, generally speaking, the variables have the meaning described in the Summary Section of the present application for patent. A person of ordinary skill in the art of organic synthesis will nevertheless readily understand that depending on the nature of the substituents designated R₁, R₂ and R₁₀ certain groups may need to be protected for the performance of the reduction step.

The substituted “3-hydroxyl-propyl amide” compounds can, generally speaking, be synthesized as described below in the following General Reaction Scheme 1 and General Reaction Scheme 2.

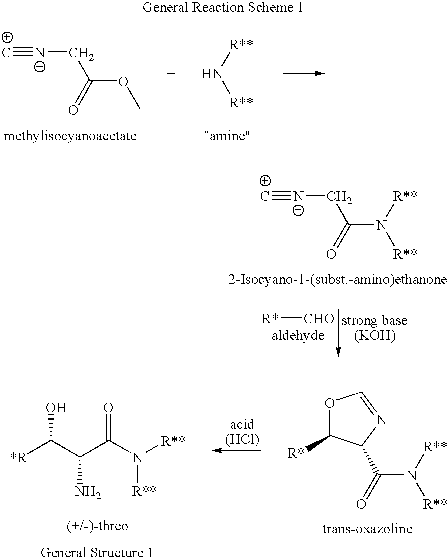

Thus, in accordance with General Scheme 1, methyl isocyanoacetate (or ethyl isocyanoacetate available commercially) is reacted with an “amine” which includes the R₁ and R₂ groups to provide the 2-isocyanoacetic acid amide derivative shown in General Reaction Scheme 1. Typical examples for the amines used in the reaction are pyrrolidine, piperidine, azetidine, morpholine, 2,5-dihydro-1H-pyrrole, dialkylamines such as diethylamine, 3-fluoro-, 3,3-difluoro or 3-hydroxy substituted pyrrolidines. The 2-isocyanoacetic acid amide derivative is then reacted in methanol in the presence of base (such as KOH) with an “aldehyde” which includes the R₁₀ group to provide a trans “oxazoline” with high diastereoselectivity (trans:cis ratios generally >97:3) as shown in General Reaction Scheme 1. The trans oxazoline is then treated in methanol with a strong acid, such as HCl, to open the ring and to provide the threo-3-substituted-3-hydroxy-2-amino-propionic acid amide intermediates (with a threo:erythro ratios generally >97:3) as shown in General Reaction Scheme 1.

Compounds of Formula 1 and or of General Structure 1, where the amino group of formula NHR₁R₂ is a weaker nucleophile, such as indoline, thiomorpholine and the like, can be made as illustrated in Reaction Scheme 2 for the synthesis of intermediate compounds (±)-threo-2-amino-3-hydroxy-1-(indolin-1-yl)-3-(pyridin-4-yl)propan-1-one dihydrochloride Compound 243 and (±)-threo-2-amino-3-hydroxy-1-(thiazolidin-3-yl)-3-(pyridin-4-yl)propan-1-one dihydrochloride Compound 242.

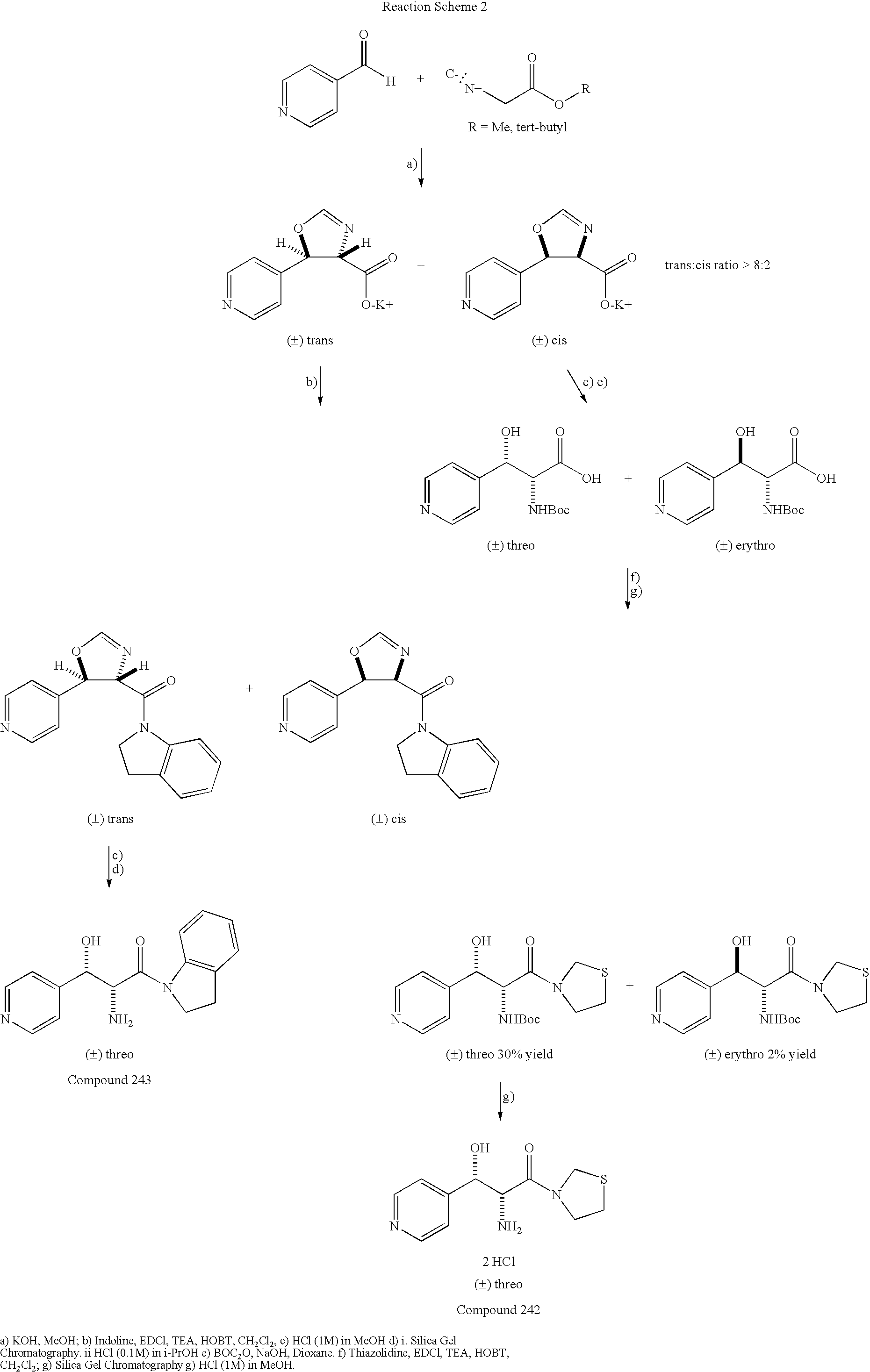

In Reaction Scheme 2 EDCI stands for 1-(3-dimethylaminopropyl)-ethylcarbodiimide hydrochloride; HOBT stands for 1-hydroxybenzotriazole; BOC₂O stands for di-t-butyl-dicarbonate and TEA stands for triethylamine. Compounds 242 and 243 can be reduced, as illustrated in General Synthetic Scheme A to provide compounds of the invention.

Another general synthetic route may follow in general terms the synthesis of Compound 1, Compound 2 and Compound 3, specifically described in detail in the experimental section below, modified with such modifications which in light of the present disclosure will become readily apparent to a person of ordinary skill in the art.

Isomerically pure and/or enantiomerically pure compounds and further derivatives of the 3-substituted-3-hydroxy-2-amino-propionic acid amide intermediates or of the substituted 1-hydroxy propylamines of the invention are obtained by separation techniques and reactions which, per se, are well known to the synthetic chemist. Some of the typical separation techniques and reactions are generally described below.

Separation of threo and erythro isomers, when both are formed in the reactions leading to the compounds of the invention, can typically be performed by chromatographic methods. The chromatographic separation may occur the level of the substituted 3-hydroxyl-propionic acid amide intermediate compounds or at the level of the substituted 1-hydroxyl propyl amine compounds of the invention.

The more abundantly formed threo isomers can also be converted into the erythro isomers by oxidizing to the ketone level the hydroxyl group in the 3 position of the propanoic acid moiety and subsequently reducing the resulting ketone to the hydroxyl level in the intermediate 3-substituted-3-hydroxy-2-amino-propionic acid amide compounds or in the compounds of the invention. Separation of enantiomeric mixtures can be performed on Chiralpack columns which are well known in the art.

The amino function in the 2-position of the propyl amine moiety is, generally speaking, more reactive towards acylation and carbamoylation than the hydroxyl group in the 1 position. Therefore, acylated derivatives of the 2-amino function can be prepared by using acyl chlorides such as acetyl chloride and hexanoyl chloride. Or the 1-hydroxy and 2-amino groups of the compounds of the invention can be acylated in the same reaction. Carbamate derivatives of the 2-amino function can be obtained by using chloroformates, such as benzylchloroformate. A tertiary butyl carbamoyl function or benzyl-carbamoyl function can also serve as a removable protecting group of the 2-amino function.

Alkylation of the 2-amino function can be performed by condensing the compound bearing the 2-NH₂ group with an aldehyde to obtain a Schiff base intermediate which can be reduced, without isolation, to provide the N-alkyl, arylalkyl or heteroaryl-alkyl compounds of the invention.

### Detailed Description of the Synthesis of Preferred Compounds (Experimental)

### Preparation of D-threo-2-amino-3-morpholino-1-phenylpropan-1-ol dihydrochloride Compound 4

### (R)-Methyl 1-((S)-1-phenylethyl)aziridine-2-carboxylate EBE 06044B

To solution of methyl 2,3-dibromopropionate (25 mL, 198 mmol) in toluene at 5° C. was added triethylamine (55 mL, 0.39 mmol) in toluene (100 mL). After stirring for 5 min (S)-(1)-phenethylamine (25 mL, 198 mmol) in toluene (100 mL) was added dropwise. The suspension was refluxed for 3 h and allowed to cool down, filtered and the volatiles were evaporated under reduced pressure to give a residue that was purified by column chromatography (950 g of silica gel) with a gradient of 0-20% EtOAc in cyclohexane to yield to (S)-methyl 1-((S)-1-phenylethyl)aziridine-2-carboxylate EBE 06044A as a yellow oil (17.31 g, 43% yield) and (R)-methyl 1-((S)-1-phenylethyl)aziridine-2-carboxylate EBE 06044B as a yellow oil (15.14 g, 37% yield).

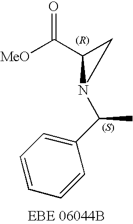

MW: 205.3; Yield EBE 06044B: 37%; Yellow Oil. Yield: EBE 06044A: 43%, Yellow Oil. R_{f}: EBE 06044A=0.5; R_{f}: EBE 06044B=0.35 (EtOAc:cyclohexane=25:75). ¹H-NMR (CDCl₃,) EBE 06044A: 1.47 (d, 3H, J=6.6 Hz, CH₃), 1.60 (d, 1H, J=6.4 Hz, CH), 2.13 (d, 1H, J=2.6 Hz), 2.21 (dd, 1H, J=3.2 Hz, J=6.4 Hz), 2.54 (q, 1H, J=6.6 Hz), 3.75 (s, 3H, OCH₃) 7.23-7.40 (m, 5H, ArH). ¹H-NMR (CDCl₃, δ): EBE 06044B: 1.46 (d, 3H, J=6.6 Hz, CH₃), 1.79 (d, 1H, J=6.6 Hz, CH), 2.08 (d, 1H, J=3.11 Hz, 6.6 Hz), 2.34 (dd, 1H, J=3.1 Hz, J=1.0 Hz), 2.56 (q, 1H, J=6.6 Hz), 3.67 (s, 3H, OCH₃) 7.24-7.36 (m, 5H, ArH). ¹³C-NMR (CDCl₃, δ): EBE 06044B: 23.5, 35.0, 36.9, 52.2, 69.8, 126.5, 127.2, 128.5, 143.6, 171.1. HPLC: Method A, detection at 254 nm, EBE 06044B RT=6.11 min, peak area 92.9%.

### ((R)-1-((S)-1-Phenylethyl)aziridin-2-yl)methanol EBE 06046

A 250 mL round bottom flask was charged with anhydrous THF (100 mL) and LiAlH₄ (2.77 g, 73.1 mmol). While the suspension is stirred at 0° C., a solution of (S)-methyl 1-((S)-1-phenylethyl)aziridine-2-carboxylate EBE 06044B (10.0 g, 48.7 mmol) in THF (50 mL) was added dropwise over 20 min The dropping funnel was washed with THF (2×3 mL) and allowed to react 20 min at 0° C. Maintaining the reaction mixture at 0° C., a solution of KOH (10%, 20 mL) was added dropwise for 20 min (caution the reaction is exothermic). The mixture was stirred for 0.5 h at 25° C. and the white precipitate removed by filtration through a celite pad that was washed with diethyl ether (30 mL). The combined organic filtrates were washed with NaH₂PO₄ and the aqueous layer was extracted with Et₂O (3×30 mL). The combined organic phase were dried with Na₂SO₄ and concentrated to give ((R)-1-(S)-1-phenylethyl)aziridin-2-yl)methanol EBE 06046 as a white solid (10.4 g, 90% yield).

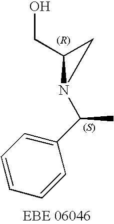

MW: 177.2; Yield: 90%; White Solid; Mp (° C.): 37.7. ¹H-NMR (CDCl₃, δ): 1.43 (d, 3H, J=6.6 Hz, CH₃), 1.49 (d, 1H, J=6.5 Hz, CH), 1.65-1.71 (m, 1H, CH), 1.92 (d, 1H, J=3.5 Hz, NCH), 2.26 (s, 1H, OH), 2.53 (q, 1H, J=6.6 Hz, NCH), 3.32-3.37 (m, 1H, OCH₂), 3.56 (m, 1H, OCH₂), 7.23-7.35 (m, 5H, ArH). ¹³C-NMR (CDCl₃, δ): 22.9, 31.4, 39.3, 62.5, 69.4, 126.6, 127.3, 128.6, 144.5.

### (R)-1-((S)-1-Phenylethyl)aziridine-2-carbaldehyde EBE 06048

A three neck, 250 mL round bottom flask was equipped with a low temperature thermometer and two (2) equalizing dropping funnels. One of these was connected to a nitrogen line and charged with a solution of ((R)-1-((S)-1-phenylethyl)aziridin-2-yl)methanol EBE 06046 (7.0 g, 39.5 mmol) in CH₂Cl₂ (75 mL), the other was charged with a solution of DMSO (9.25 g, 118.5 mmol) in CH₂Cl₂ (11 mL). To a solution of oxalyl chloride (7.5 g, 59.3 mmol) in CH₂Cl₂ (90 mL) under N₂ at −78° C., the DMSO solution was added dropwise during 20 min and stirred for 20 min. EBE 06046 (7.0 g, 39.5 mmol) in CH₂Cl₂ (75 mL) was added dropwise over 50 min. then the dropping funnel was charged with DIEA (42.6 mL, 237 mmol) in CH₂Cl₂ (10 mL) and the reaction mixture was stirred for 30 min at −45° C. The DIEA solution was added over 5 min with the reaction mixture at −78° C. and the reaction was allowed to warm to room temperature. The reaction mixture was washed with H₂O (3×50 mL), dried over MgSO₄, filtered, evaporated. The crude product obtained was purified by column chromatography on silica with a gradient of 0-20% [v/v] EtOAc in cyclohexane to give (R)-1-((S)-1-phenylethyl)aziridine-2-carbaldehyde EBE 06048 as a yellow oil (5.59 g, 81% yield).

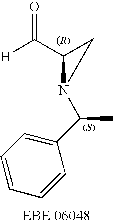

MW: 175.2; Yield: 81%; Yellow Oil. R_{f}: EBE 06048: 0.3 (EtOAc:cyclohexane=20:80). ¹H-NMR (CDCl₃, δ): 1.47 (d, 3H, J=6.6 Hz, CH₃), 1.94 (d, 1H, J=6.7 Hz, NCH₂), 2.08 (dt, J=2.9 Hz, J=6.4 Hz, NCH), 2.37 (d, 1H, J=2.6 Hz, NCH₂), 2.61 (q, 1H, J=6.6 Hz, NCH), 7.20-7.38 (m, 5H, ArH), 8.92 (d, 1H, J=6.2 Hz). ¹³C-NMR (CDCl₃, δ): 22.7, 32.1, 43.2, 68.1, 125.5, 126.5, 127.6, 142.4, 198.7.

### (R)-Phenyl((R)-1-(S)-1-phenylethyl)aziridin-2-yl)methanol EBE 06066

To a solution of bromobenzene (4.93 g, 31.4 mmol) in THF 125 mL under nitrogen at −78° C. was added t-BuLi (1.7 M in pentane, 50 mL). The mixture was stirred for 0.5 h at room temperature. The mixture was cooled down to −78° C. and a solution of (R)-1-((S)-1-phenylethyl)aziridine-2-carbaldehyde EBE 06048 (2.5 g, 14.3 mmol) in THF (16.7 mL) at −78° C. was added dropwise. The reaction mixture was treated with H₂O (20 mL), the organic layer was separated and the aqueous phase was extracted with EtOAc. The combined organic layers were dried over MgSO₄, filtered and concentrated in vacuo to give a residue that was purified by column chromatography using a gradient of 0-20% [v/v] EtOAc in cyclohexane to give (R)-phenyl((R)-1-((S)-1-phenylethyl)aziridin-2-yl)methanol EBE 06066 (3.13 g, 86% yield).

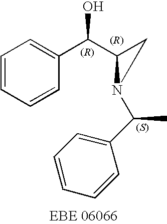

MW: 253.3; Yield: 86%. R_{f}: =0.3 (EtOAc:cyclohexane=20:80). ¹H-NMR (CDCl₃, δ): 1.47 (d, 3H, J=6.6 Hz, CH₃), 1.57 (d, 1H, J=6.5 Hz, CH), 1.79 (dt, 1H, J=3.5 Hz, J=8.7 Hz, CH), 2.04 (d, 1H, J=3.5 Hz, OCH), 2.35 (bs, 1H, OH), 2.53 (q, 1H, J=6.5 Hz, CH), 4.23 (d, 1H, J=5.7 Hz, OCH), 7.07-7.13 (m, 2H, ArH), 7.16-7.20 (m, 3H, ArH), 7.24-7.34 (m, 5H, ArH). ¹³C-NMR (CDCl₃, δ): 22.4, 32.0, 44.6, 69.4, 74.1, 125.8 (2×C), 126.9 (2×C), 127.3, 127.6, 128.2 (2×C), 128.7 (2×C), 142.0, 144.2. [α]²²_{D}=−71.53 (c=0.59, CHCl₃).

### D-threo-2-((S)-1-Phenylethylamino)-3-morpholino-1-phenylpropan-1-ol dihydrochloride Compound 5

To a solution of (R)-phenyl((R)-1-(S)-1-phenylethyl)aziridin-2-yl)methanol EBE 06066 (1.5 g, 5.92 mmol) in CH₃CN (19 mL) at RT was added iodotrimethylsilane (3.55 g, 17.8 mmol). The solution was stirred for 2 h and morpholine (1.032 g, 11.84 mmol) was added. After 2 h at reflux, the reaction mixture was treated with HCl (1M) to reach pH=1 and stirred for 10 min. After a slow addition of NaHCO₃ to reach pH=9, the product was extracted with EtOAc, dried over Na₂SO₄, filtered to give after evaporation a crude brown oil that was purified by column chromatography using a gradient of 0-20% [v/v] MeOH in EtOAc to give D-threo-2-((S)-1-phenylethylamino)-3-morpholino-1-phenylpropan-1-ol EBE 06068A (0.831 g, 42%) as a pale brown solid. To a solution of D-threo-2-((S)-1-phenylethylamino)-3-morpholino-1-phenylpropan-1-ol EBE 06068A (0.100 g, 0.294 mmol) in ethanol (1 mL) was added a solution of HCl (0.8 M, 0.816 mL) in EtOH. Evaporation of the volatiles afforded to D-threo-2-((S)-1-phenylethylamino)-3-morpholino-1-phenylpropan-1-ol dihydrochloride Compound 5 as white solid (0.125 g, 100%).

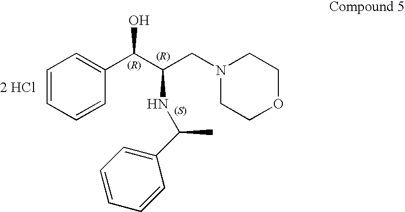

MW: 412.37; Yield: 42%; White Solid; Mp (° C.): 157.2 (dec). R_{f}: 0.3 (MeOH:EtOAc=20:80) EBE 06068A. ¹H-NMR (CD₃OD, δ): 1.19 (t, 2H, J=7.0 Hz, NCH₂), 1.71 (d, 3H, J=6.8 Hz, CH₃), 3.45 (m, 2H, J=7.1 Hz, NCH₂), 3.62 (q, 2H, J=7.1 Hz, N—CH₂), 3.97 (t, 4H, J=4.5 Hz, OCH₂), 4.06 (m, 1H, CH—N), 4.75 (q, 1H, J=6.8 Hz, CH—N), 5.21 (d, 1H, J=5.1 Hz, CH—O), 7.44-7.56 (m, 10H, ArH). MS-ESI m/z (% rel. Int.): 341.1 ([MH]⁺, 20). ¹³C-NMR (CD₃OD, δ): 24.4, 54.5 (2×C), 55.5, 55.9, 60.0, 67.0 (2×C), 75.6, 126.3 (2×C), 126.5 (2×C), 127.0, 127.1, 128.1 (2×C), 128.5 (2×C), 142.2, 145.3. HPLC: Method A, detection at 254 nm, Compound 5 RT=4.41 min, peak area 99%.

### Threo-2-Amino-3-morpholino-1-phenylpropan-1-ol dihydrochloride Compound 4

To a solution of D-threo-2-((S)-1-phenylethylamino)-3-morpholino-1-phenylpropan-1-ol EBE 06068A (0.400 g, 1.17 mmol) in MeOH (6 mL) at RT was added acetic acid (0.133 mL, 2.35 mmol). The reaction vessel was flushed with nitrogen and Pd(OH)₂ (25% weight, 0.150 g) was added. The nitrogen atmosphere was exchanged with hydrogen using three cycle of vacuum and hydrogen addition using a balloon of hydrogen. After stirring for 16 h under hydrogen the reaction mixture was filtrated through celite to give EBE 06070A the acetate salt of (2R)-amino-3-morpholin-4-yl-(1R)-phenyl-propan-1-ol (0.279 g, 98% yield). To as solution of EBE 06070A the acetate salt of (2R)-amino-3-morpholin-4-yl-(1R)-phenyl-propan-1-ol (0.100 g, 0.338 mmol) in ethanol (1 mL) was added a solution of HCl (0.8 M, 0.930 mL) in EtOH. Evaporation of the volatiles afforded to D-threo-2-amino-3-morpholino-1-phenylpropan-1-ol dihydrochloride Compound 4 (0.104 g, 100% yield) as an off white solid. (Adapted from Shin, S-H.; Han, E. Y.; Park, C. S.; Lee, W. K.; Ha, H.-J. Tetrahedron Asymmetry, 2000, 11, 3293-3301).

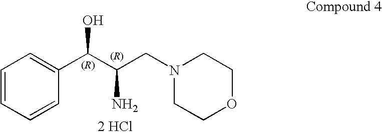

MW: 309.23; Yield: 99%; Off White Solid; Mp (° C.): 183.4. ¹H-NMR (CD₃OD, δ): 3.30-3.77 (m, 6H, CH₂N), 3.92-4.05 (m, 4H, CH₂O), 4.05-4.16 (m, 1H, CH), 4.85-4.98 (m, 1H, CH), 7.35-7.60 (m, 5H, ArH). ¹³C-NMR (CD₃OD): 53.2, 58.3, 58.5 (2×C), 64.9 (2×C), 72.6, 128.0 (2×C), 130.2 (2×C), 140.3. MS-ESI m/z (% rel. int.): 237.1 (100, [MH]⁺). HPLC: Isocratic 10% CH₃CN in H₂O (pH 10, [NH₄OH]=5 mM), detection UV 254 nm, Compound 4 RT=6.63 min, peak area 97.3%. [α]²²_{D}=−10.7 (c=1.00, MeOH).

### Preparation of Benzyl L-threo-1-hydroxy-3-morpholino-1-phenylpropan-2-ylcarbamate hydrochloride Compound 1

### Benzyl (S)-3-hydroxy-1-oxo-1-phenylpropan-2-ylcarbamate TTA 08010B

To a stirred solution of Z-L-Ser-OH (6.00 g, 25.08 mmol) in 32 mL of anhydrous THF at 0° C. under nitrogen was added dropwise 1 M phenylmagnesium bromide in THF (32 mL, 200 mmol). (The symbol Z designates a benzylcarbamoyl group). The mixture was stirred 15 h at RT under nitrogen. A solution of 2 M HCl (100 mL) was slowly added at 0° C. and the mixture was partitioned between ethyl acetate (750 mL) and acidic water. The organic layer was washed with water (2×20 mL), 1 N aqueous sodium bicarbonate (2×20 mL), brine (2×20 mL) and dried over MgSO₄. After removing ethyl acetate by evaporation at 30-35° C., the crude product (4.50 g, 60% yield) was cristallized in a mixture of ethyl acetate:hexane=25 mL:20 mL to give benzyl (S)-3-hydroxy-1-oxo-1-phenylpropan-2-ylcarbamate TTA 08010B as a white solid (1.40 g, 20% yield).

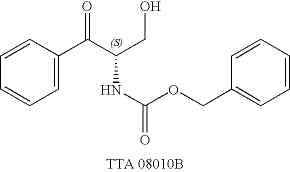

MW: 299.32; Yield: 20%; White Solid; Mp (° C.): 106.5. R_{f}: 0.75 (CH₂Cl₂:MeOH=9:1). ¹H-NMR (CDCl₃, δ): 2.78 (s, 1H, OH), 3.85-3.93 (m, 1H, CH₂O), 4.00-4.09 (m, 1H, CH₂O), 5.14 (s, 2H, ArCH₂O), 5.40 (t, 1H, J=3.3 Hz, CH), 6.17 (d, 1H, J=6.4 Hz, NH), 7.35 (s, 5H, ArH), 7.49 (t, 2H, J=7.60 Hz, ArH), 7.62 (t, 1H, J=7.1 Hz, ArH), 8.99 (t, 2H, J=7.6 Hz, ArH). ¹³C-NMR (CDCl₃, δ): 58.3, 64.6, 67.3, 128.1, 128.3, 128.6, 128.7, 129.0, 134.1, 136.0, 156.6, 196.6. MS-ESI m/z (% rel. Int.): 300.1 ([MH]⁺, 5), 256.1 (100). HPLC: Method A, detection UV 254 nm, TTA 08010B RT=5.40 min, peak area 98.5%. [α]²²_{D}=−5.8 (c=1.00, MeOH).

### Benzyl L-threo-1,3-dihydroxy-1-phenylpropan-2-ylcarbamate TTA 08012

To a stirred solution of benzyl (S)-3-hydroxy-1-oxo-1-phenylpropan-2-ylcarbamate TTA 08010B (1.40 g, 4.70 mmol) in 28 mL of anhydrous THF at −78° C. under nitrogen was added slowly dropwise 1 M DIBAL-H in hexane (18.8 mL, 18.80 mmol). The mixture was stirred 2 h at −78° C. then 1.5 h at RT. A solution of 2 M HCl (35 mL) was slowly added at −20° C. and the mixture was partitioned between ethyl acetate (750 mL) and acidic water. The organic phase was washed with water (2×20 mL), brine (2×20 mL) and dried over MgSO₄. After removing ethyl acetate by evaporation at 30-35° C., the crude product was purified by column chromatography on silica (CH₂Cl₂:MeOH=98:2 to 97:3) to give benzyl L-threo-1,3-dihydroxy-1-phenylpropan-2-ylcarbamate TTA 08012 as a white solid (1.10 g, 78% yield).

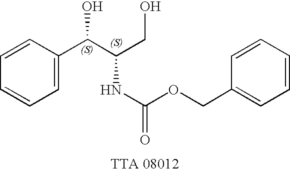

MW: 301.34; Yield: 78%; White Solid; Mp (° C.): 102.5. R_{f}: 0.30 (CH₂Cl₂:MeOH=95/5). ¹H-NMR (CDCl₃, δ): 3.08 (t, 1H, J=5.0 Hz, OH), 3.59 (d, 1H, J=3.1 Hz, OH), 3.64-3.78 (m, 2H, CH₂O), 3.80-3.89 (m, 1H, CH), 4.95 (s, 2H, ArCH₂O), 5.57 (d, 1H, J=8.3 Hz, NH), 7.17-7.38 (m, 10H, ArH). ¹³C-NMR (CDCl₃, δ): 57.5, 63.6, 66.9, 73.8, 126.0, 127.8, 127.9, 128.1, 128.5, 128.6, 136.2, 141.0, 156.9. MS-ESI m/z (% rel. Int.): 302.0 ([MH]⁺, 5); 132.0 (100). HPLC: Method A, detection UV 254 nm, TTA 08012 RT=5.00 min, peak area 99.5%. [α]²²_{D}=+39.4 (c=1.00, MeOH).

### Benzyl threo-1-hydroxy-3-morpholino-1-phenylpropan-2-ylcarbamate hydrochloride Compound 1

To a stirred solution of benzyl L-threo-1,3-dihydroxy-1-phenylpropan-2-ylcarbamate TTA 08012 (1.00 g, 3.30 mmol) in 13 mL of pyridine at −10° C. was added dropwise methanesulfonyl chloride (0.27 mL, 3.50 mmol). The mixture was stirred 6 h at 20° C. under nitrogen. Pyridine was removed by evaporation at 30-35° C. and the residue was partitioned between ethyl acetate (250 mL) and 0.1 N HCl (20 mL). The organic phase was washed with water (20 mL), brine (20 mL), dried over MgSO₄ and evaporated to give after drying L-threo-1-hydroxy-3-methanesulfonyl-1-phenylpropan-2-ylcarbamate TTA 08014 (1.25 g, 65% yield).

To a stirred solution of crude benzyl L-threo-1-hydroxy-3-methanesulfonyl-1-phenylpropan-2-ylcarbamate TTA 08014 (1.25 g, 3.30 mmol) in 6 mL of DMF at RT was added morpholine (1.2 mL, 13.20 mmol). The mixture was stirred 15 h at 50° C. under nitrogen. DMF was evaporated and the residue was partitioned between ethyl acetate (250 mL) and 1 N aqueous sodium bicarbonate (20 mL). The organic phase was washed with water (20 mL), brine (20 mL) and dried over MgSO₄. After evaporation the crude product was purified by column chromatography on silica (CH₂Cl₂:MeOH=98:2 to 97:3) to give benzyl L-threo-1-hydroxy-3-morpholino-1-phenylpropan-2-ylcarbamate as an oil (380 mg, 31% yield). The hydrochloride salt was obtained from 100 mg of the free base in diethylether at 0° C. using a solution 0.3 M HCl in diethylether. The precipitate was filtered and dry to give benzyl L-threo-1-hydroxy-3-morpholino-1-phenylpropan-2-ylcarbamate hydrochloride Compound 1 as a white solid (70 mg, 65% yield).

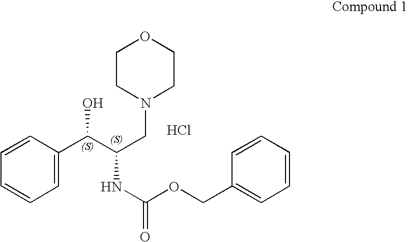

MW: 406.90; Yield: 20%; White Solid; Mp (° C.): 144.5. R_{f}: 0.40 (CH₂Cl₂:MeOH=95:5). ¹H-NMR (CD₃OD, δ): 3.14-3.77 (m, 6H, CH₂N), 3.70-4.07 (m, 4H, CH₂O), 4.30-4.33 (m, 1H, CH), 4.90-5.06 (m, 3H, CH, ArCH₂O), 7.20-7.43 (m, 10H, ArH). ¹³C-NMR (CD₃OD, δ): 51.2, 51.8, 53.2, 59.3, 63.2, 66.3, 72.5, 125.8, 127.2, 127.3, 127.5, 127.8, 127.9. MS-ESI m/z (% rel. Int.): 371.0 ([MH]⁺, 100). HPLC: Method A, detection UV 254 nm, Compound 1 RT=4.40 min, peak area 96.5%. [α]²²_{D}=+13.9 (c=1.00, MeOH).

### Preparation of threo-2-amino-3-morpholino-1-phenylpropan-1-ol dihydrochloride Compound 2

To a stirred solution of benzyl L-threo-1-hydroxy-3-morpholino-1-phenylpropan-2-ylcarbamate (Compound 1, 0.26 g, 0.70 mmol) in 20 mL of MeOH at RT was added Pd—C 10% (140 mg). The mixture was satured with hydrogen and stirred for 24 h at RT under hydrogen atmosphere (balloon). The catalyst Pd—C 10% was removed by filtration on celite and the solution was evaporated. The crude product was purified by column chromatography on silica (CH₂Cl₂:MeOH:NH₄OH=79:20:1 to 75:20:5) to give L-threo-2-amino-3-morpholino-1-phenylpropan-1-ol as an oil (100 mg, 60% yield). The hydrochloride salt was obtained from 83 mg of the free base in diethylether at 0° C. using 0.3 M HCl in diethylether. After precipitation in diethylether, filtration and drying L-threo-2-amino-3-morpholino-1-phenylpropan-1-ol dihydrochloride Compound 2 was obtained as a white solid (80 mg, 74% yield).

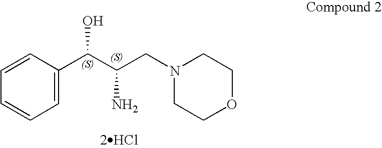

MW: 309.23; Yield: 44%; White Solid; Mp (° C.): 166.4-170.9. R_{f}: 0.20 (CH₂Cl₂:MeOH=9:1). ¹H-NMR (CD₃OD, δ): 3.30-3.77 (m, 6H, CH₂N), 3.92-4.05 (m, 4H, CH₂O), 4.05-4.16 (m, 1H, CH), 4.85-4.98 (m, 1H, CH), 7.35-7.60 (m, 5H, ArH). ¹³C-NMR (CD₃OD, δ): 53.1, 54.9, 58.5, 64.8, 72.6, 127.2, 128.0, 130.2, 140.3. MS-ESI m/z (% rel. Int.): 237.0 ([MH]⁺, 100). HPLC: Method A, detection UV 254 nm, Compound 2 RT=0.90 min, peak area 98.0%. [α]²²_{D}=+10.8 (c=1.00, MeOH), free base: [α]²²_{D}=−6.1 (c=0.25, CHCl₃).

### Preparation of benzyl L-threo-1-acetoxy-3-morpholino-1-phenylpropan-2-ylcarbamate hydrochloride Compound 3

### Benzyl L-threo-1-acetoxy-3-morpholino-1-phenylpropan-2-ylcarbamate hydrochloride Compound 3

To a stirred solution of benzyl L-threo-1-hydroxy-3-morpholino-1-phenylpropan-2-ylcarbamate hydrochloride (Compound 1, 0.510 g, 1.25 mmol) in 30 mL of CHCl₃ at RT were added slowly triethylamine (700 μL, 5.00 mmol) and acetyl chloride (145 μL, 2.00 mmol). The mixture was stirred 10 h at RT under nitrogen and partitioned between a mixture of ice-water (20 mL) and CH₂Cl₂ (100 mL). The organic layer was washed with brine (20 mL) and dried over MgSO₄. After evaporation the crude product was purified by column chromatography on silica (CH₂Cl₂:MeOH=99.5:0.5 to 98:2) to give benzyl L-threo-1-acetoxy-3-morpholino-1-phenylpropan-2-ylcarbamate as an oil (0.420 g, 81% yield).

The hydrochloride salt was obtained from 45 mg of the free base in diethylether at 0° C. using a solution of 0.3 M HCl in diethylether. The precipitate was filtered and dry to give benzyl L-threo-1-acetoxy-3-morpholino-1-phenylpropan-2-ylcarbamate hydrochloride Compound 3 as a white solid (40 mg, 82% yield).

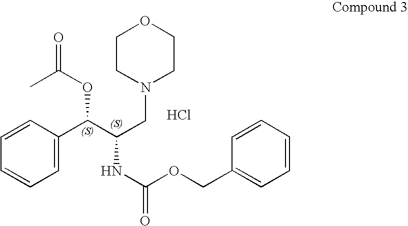

MW: 448.94; Yield: 66%; White Solid; Mp (° C.): 69.9. R_{f}: 0.70 (CH₂Cl₂:MeOH=95:5). ¹H-NMR (CD₃OD, δ): 2.10 (s, 3H, CH₃), 3.14-3.44 (m, 4H, CH₂N), 3.70-4.00 (m, 4H, CH₂O), 4.51-4.53 (m, 1H, CH), 4.90-5.13 (m, 2H, ArCH₂O), 5.89 (d, 1H, CH), 7.28-7.48 (m, 10H, ArH). ¹³C-NMR (CD₃OD, δ): 20.8, 52.0, 52.6, 59.7, 64.6, 68.0, 76.5, 127.7, 129.0, 129.2, 129.5, 129.8, 137.9, 158.7, 171.3. MS-ESI m/z (% rel. Int): 413.0 ([MH]⁺, 100). HPLC: Method A, detection UV 254 nm, Compound 3 RT=4.70 min, peak area 98.5%.

### Preparation of DL-threo-2-(Decanamido)-1-(4-methoxyphenyl)-3-(pyrrolidin-1-yl)propyl decanoate Compound 10

### 2-Isocyano-1-(pyrrolidin-1-yl)ethanone BLE 04098

To stirred and cooled (0° C.) methyl isocyanoacetate (96% technical grade, 5.0 g, 47.8 mmol) was slowly added in 0.75 h pyrrolidine (6.5 mL, 78 mmol). The mixture was stirred for 1.5 h with continued cooling and then concentrated. The resulting oil was co-evaporated twice from CH₂Cl₂:hexane to remove residual pyrrolidine. 2-Isocyano-1-(pyrrolidin-1-yl)ethanone BLE 04098 was obtained as a yellow solid (6.85 g, 98% yield) and used in the next step without purification.

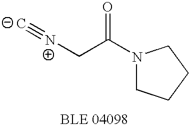

MW: 138.17; Yield: 98%; yellow solid; Mp (° C.)=73.9. ¹H-NMR (CDCl₃, δ): 1.81-2.08 (m, 4H, 2×CH₂), 3.35-3.45 (m, 2H, —NCH₂), 3.50-3.60 (m, 2H, —NCH₂), 4.23 (s, 2H, CH₂CO).

### Trans-(4,5-dihydro-5-(pyridin-3-yl)oxazol-4-yl)(pyrrolidin-1-yl)methanone BLE 04110B

### trans-(4,5-Dihydro-5-(4-methoxyphenyl)oxazol-4-yl)(pyrrolidin-1-yl)methanone SLA 07074

To a stirred and cooled (0° C.) solution of potassium hydroxide (0.37 g, 6.57 mmol) in methanol (30 mL) was added a mixture of 4-methoxy-benzaldehyde (0.88 mL, 7.23 mmol) and 2-isocyano-1-(pyrrolidin-1-yl)ethanone BLE 04098 (1.0 g, 6.57 mmol). The solution was stirred 4 h with continued cooling and then concentrated. The residue was partitioned between ethyl acetate and water. The organic layer was combined with additional ethyl acetate extracts, washed with aqueous sodium chloride and dried over MgSO₄. Concentration afforded a crude product as a glassy solid. Flash chromatography over silica (ethyl acetate) yielded to trans-(4,5-dihydro-5-(4-methoxyphenyl)oxazol-4-yl)(pyrrolidin-1-yl)methanone SLA 07074 as a pale yellow solid (1.2 g, 90.5%).

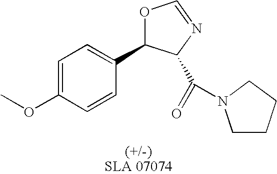

MW: 274.32; Yield: 90.5%; pale yellow solid; Mp (° C.): 91.2. R_{f}: 0.30 (EtOAc). ¹H-NMR (CDCl₃, δ): 1.75-2.08 (m, 4H, 2×CH₂), 3.40-3.58 (m, 3H, CH₂N), 3.52 (s, 3H, CH₃O), 3.88-3.98 (m, 1H, CH₂N), 4.59 (dd, 1H, J=7.6 Hz, J=2.2 Hz, CH—N), 6.06 (d, 1H, J=7.6 Hz, CH—O), 6.90 (d, 2H, J=8.7 Hz, ArH), 7.01 (d, 1H, J=2.2 Hz, CH═N), 7.25 (d, 2H, J=8.7 Hz, ArH). MS-ESI m/z (% rel. Int.): 275.1 ([MH]⁺, 10), 247.1 (100). HPLC: Method A, detection UV 280 nm, SLA 07074 RT=5.2 min, peak area 92%.

### DL-threo-2-Amino-3-hydroxy-3-(4-methoxyphenyl)-1-(pyrrolidin-1-yl)propan-1-one hydrochloride SLA 07078

To a stirred solution of trans-(4,5-dihydro-5-(4-methoxyphenyl)oxazol-4-yl)(pyrrolidin-1-yl)methanone SLA 07074 (1.61 g, 5.93 mmol) in methanol (13 mL) was added hydrochloric acid (1 mL). After heating at 50° C. for 3 h the mixture reaction was concentrated and the resulting yellow oil was co-evaporated twice with ethyl acetate before solidifying. Trituration (ethyl acetate) and drying afforded DL-threo-2-amino-3-hydroxy-3-(4-methoxyphenyl)-1-(pyrrolidin-1-yl)propan-1-one hydrochloride SLA 07078 as a white solid (1.64 g, 93%).

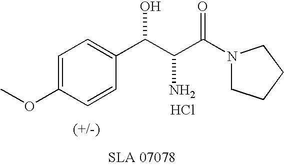

MW: 300.78; Yield: 93%; white Solid; Mp (° C.): 177.0. ¹H-NMR (CD₃OD, δ): 1.32-1.50 (m, 1H, CH₂), 1.50-1.88 (m, 3H, CH₂), 2.15-2.28 (m, 1H, CH₂N), 3.15-3.42 (m, 4H, 2×CH₂N), 3.79 (s, 3H, CH₃O), 4.06 (d, 1H, J=9.2 Hz, CH—N), 4.78 (d, 1H, J=9.2 Hz, CHO), 6.94 (d, 2H, J=8.5 Hz, ArH), 7.34 (d, 2H, J=8.5 Hz, ArH). ¹³C-NMR (CD₃OD, δ): 24.8, 26.6, 47.2, 47.6, 55.9, 59.6, 73.9, 115.0 (2×C), 128.9 (2×C), 132.5, 161.7, 166.4.

### DL-threo-2-Amino-1-(4-methoxyphenyl)-3-(pyrrolidin-1-yl)propan-1-ol Compound 9

To a stirred suspension of DL-threo-[5-(4-methoxy-phenyl)-4,5-dihydro-oxazol-4-yl]-pyrrolidin-1-yl-methanone SLA 07078 (1.61 g, 5.35 mmol) in tetrahydrofuran (200 mL) under nitrogen atmosphere was slowly added, in two portions, lithium aluminium hydride (1.22 g, 32.12 mmol) at 0° C. The mixture reaction was stirred at RT for 17 h, and then quenched by a slow, dropwise addition of water (50 mL). The white suspension was then concentrated to remove THF and taken back up in a mixture of 300 mL CH₂Cl₂ and 1N aqueous hydrochloric acid (50 mL). The aqueous layer was basified to pH=10-11 by a slow addition of 1N aqueous sodium hydroxyde. The organic layer was removed, combined with additional CH₂Cl₂ extracts (4×200 mL) and dried over MgSO₄, filtered and evaporated. The crude product was purified by column chromatography on silica (CH₂Cl₂:MeOH:NH₃=94:05:01). After evaporation and drying, DL-threo-2-amino-1-(4-methoxyphenyl)-3-(pyrrolidin-1-yl)propan-1-ol Compound 9 was obtained (0.62 g, 46%) as a pale yellow solid.

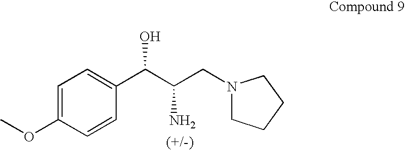

MW: 250.34; Yield: 46%; Pale Yellow Solid; MP (° C.): 77.7. R_{f}: 0.35 (CH₂Cl₂:MeOH:NH₃=94:05:01). ¹H-NMR (CDCl₃, δ): 1.65-1.87 (s, 4H, 2×CH₂), 2.40-2.90 (m, 9H, CH₂N, NH₂ & OH), 3.11-3.17 (m, 1H, CH—N), 3.81 (s, 3H, CH₃O), 4.61 (d, 1H, J=3.8 Hz, CH—O), 7.89 (d, 2H, J=8.6 Hz, ArH), 7.26 (d, 2H, J=8.5 Hz, ArH). ¹³C-NMR (CDCl₃, δ): 23.6 (2×C), 54.5, 54.7 (2×C), 55.3, 60.1, 75.9, 113.6, 127.4, 134.4, 158.8. MS-ESI m/z (% rel. Int.): 251.1 ([MH]⁺, 100).

### DL-threo-2-(Decanamido)-1-(4-methoxyphenyl)-3-(pyrrolidin-1-yl)propyl decanoate Compound 10

To a stirred solution of DL-threo-2-amino-1-(4-methoxy-phenyl)-3-pyrrolidin-1-yl-propan-1-ol Compound 9 (0.15 g, 0.60 mmol) in dichloromethane (10 mL) were added N-hydroxysuccinimide (0.07 g, 0.60 mmol), triethylamine (0.10 mL, 0.63 mmol) and decanoyl chloride (112 μL, 0.54 mmol) under nitrogen atmosphere. The mixture reaction was stirred at RT for 22 h and partitioned between methylene chloride and 1 N aqueous sodium hydroxide. The organic layer was dried over MgSO₄, filtered and evaporated. The crude product was purified by column chromatography on silica (CH₂Cl₂:MeOH=95:05). DL-threo-2-(Decanamido)-1-(4-methoxyphenyl)-3-(pyrrolidin-1-yl)propyl decanoate Compound 10 was obtained as a white oil (0.104 g, 31%).

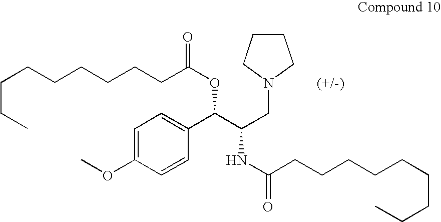

MW: 558.84; Yield: 40%; White Oil. R_{f}: 0.35 (CH₂Cl₂:MeOH=95:05). ¹H-NMR (CDCl₃, δ): 0.88 (t, 6H, J=0.7 Hz, 2×CH₃), 1.26 (s, 14H, 7×CH₂), 1.57-1.59 (m, 4H, 2×CH₂), 1.80 (m, 4H, 2×CH₂), 2.10-2.50 (m, 5H, CH₂), 2.65-2.76 (m, 5H, CH₂), 3.79 (s, 3H, CH₃O), 4.54 (m, 1H, CH—N), 5.89 (d, 1H, J=6.2 Hz, CH—O), 6.16 (d broad, 1H, J=8.8 Hz, NH), 6.85 (d, 2H, J=8.7 Hz, ArH), 7.24 (d, 2H, J=8.7 Hz, ArH). MS-ESI m/z (% rel. int.): 559.5 ([MH]⁺, 100). HPLC: Method A, detection UV 280 nm, Compound 10 RT=6.99 min, peak area 96.4%.

### N-(DL-threo-1-Hydroxy-1-(4-methoxyphenyl)-3-(pyrrolidin-1-yl)propan-2-yl)palmitamide or DL-threo-4-MeO-P4 Compound 11

To a stirred solution of DL-threo-2-amino-1-(4-methoxyphenyl)-3-(pyrrolidin-1-yl)propan-1-ol Compound 9 (015 g, 0.60 mmol) in dichloromethane (10 mL) were successively added N-hydroxysuccinimide (0.07 g, 0.60 mmol), triethylamine (0.100 mL, 0.63 mmol) and palmitoyl chloride (0.15 g, 0.54 mmol) under nitrogen atmosphere. The mixture reaction was stirred at RT for 17 h and partitioned between methylene chloride and 1N aqueous sodium hydroxide. The organic layer was dried over MgSO₄, filtered and evaporated. The crude product was purified by column chromatography on silica (CH₂Cl₂:MeOH=95:05). N-(DL-threo-1-Hydroxy-1-(4-methoxyphenyl)-3-(pyrrolidin-1-yl)propan-2-yl)palmitamide Compound 11 was obtained as a white solid (0.117 g, 40%).

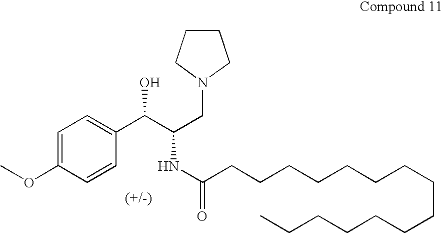

MW: 488.75; Yield: 40%; White Solid; Mp (° C.): 82.3. R_{f}: 0.35 (CH₂Cl₂:MeOH=95:05). ¹H-NMR (CDCl₃,: 0.88 (t, 3H, J=7.0 Hz, CH₃), 1.22-1.33 (m, 16H, 8×CH₂), 1.47-1.54 (m, 2H, CH₂), 1.81 (m, 4H, 2×CH₂), 2.09 (t, 2H, J=7.0 Hz, COCH₂), 2.60-2.80 (m, 4H, 2×CH₂), 2.84 (d, 2H, J=5.1 Hz, CH₂), 3.80 (s, 3H, CH₃O), 4.23 (m, 1H, CH—N), 5.00 (d, 1H, J=2.2 Hz, CH—O), 5.90 (d, 1H, J=7.4 Hz, NH), 6.87 (d, 2H, J=8.7 Hz, ArH), 7.24 (d, 2H, J=8.7 Hz, ArH). ¹³C-NMR (CDCl₃, δ): 14.1, 22.7, 23.6, 25.6, 29.1, 29.3, 29.4, 29.5, 29.7, 29.7, 31.9, 36.8, 52.3, 55.2, 57.8, 75.4, 113.7 (2×C), 127.0 (2×C), 133.1, 158.9, 173.6. MS-ESI m/z (rel. int.): 489.2 ([MH]⁺, 100). HP LC: Method A, detection UV 280 nm, Compound 11 RT=6.55 min, peak area 96.4%.

### DL-threo-2-Amino-1-(2,3-dihydrobenzo[b][1,4]dioxin-6-yl)-3-(pyrrolidin-1-yl)propan-1-ol Compound 6

### trans-(4,5-Dihydro-5-(2,3-dihydrobenzo[b][1,4]dioxin-6-yl)oxazol-4-yl)(pyrrolidin-1-yl)methanone BLE 04100

To a stirred and cooled (0° C.) solution of potassium hydroxide (0.43 mg, 7.60 mmol) in MeOH (6.5 mL) were added successively 1,4-benzodioxan-6-carboxaldehyde (1.31 g, 7.96 mmol) and 2-isocyano-1-(pyrrolidin-1-yl)ethanone BLE 04098 (1.0 g, 6.57 mmol). The solution was stirred 3 h at 0° C. and then concentrated. The residue was partitioned between EtOAc (100 mL) and water. The organic layer was combined with 2 additional EtOAc extracts (2×100 mL), washed with brine, dried over MgSO₄, filtered and evaporated. Concentration afford to a crude product which was purified by column chromatography on silica (EtOAc) to yield, after evaporation and drying, to trans-4,5-dihydro-5-(2,3-dihydrobenzo[b][1,4]dioxin-6-yl)oxazol-4-yl)(pyrrolidin-1-yl)methanone BLE 04100 as a colourless oil (1.76 g, 89% yield).

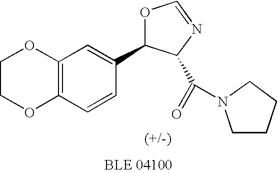

MW: 440.49; Yield: 89%; colourless oil. ¹H-NMR (CDCl₃, δ): 1.75-2.10 (m, 4H, 2×CH₂), 3.40-3.59 (m, 6H, 3×CH₂N), 3.85-4.00 (m, 1H, CHN), 4.26 (s, 4H, CH₂O), 4.59 (dd, 1H, J=7.5 Hz, J=2.2 Hz, CH—N), 6.00 (d, 1H, J=7.5 Hz, CH—O), 6.75-6.90 (m, 3H, ArH), 7.00 (d, 1H, J=2.2 Hz, CH═N).

### DL-threo-2-amino-3-(2,3-dihydrobenzo[b][1,4]dioxin-6-yl)-3-hydroxy-1-(pyrrolidin-1-yl)propan-1-one hydrochloride Compound 12

To a stirred solution of trans-4,5-dihydro-5-(2,3-dihydrobenzo[b][1,4]dioxin-6-yl)oxazol-4-yl)(pyrrolidin-1-yl)methanone BLE 04100 (1.74 g, 5.77 mmol) in methanol (15 mL) was added hydrochloric acid (1 mL). After heating at 50° C. for 3 h the mixture reaction was concentrated and the resulting yellow oil was co-evaporated twice with ethyl acetate before solidifying. Trituration (ethyl acetate) and drying afforded DL-threo-2-amino-3-(2,3-dihydrobenzo[b][1,4]dioxin-6-yl)-3-hydroxy-1-(pyrrolidin-1-yl)propan-1-one hydrochloride Compound 12 as a white solid (1.85 g, 95%).

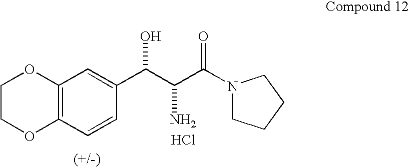

MW: 328.79; Yield: 95.0%; White Solid; Mp (° C.): 176.2. ¹H-NMR (CD₃OD, δ): 1.42-1.58 (m, 1H, CH₂), 1.58-1.70 (m, 1H, CH₂), 1.70-1.88 (m, 2H, CH₂), 3.20-3.45 (m, 4H, N—CH₂), 4.06 (d, 1H, J=9.1 Hz, CH—N), 4.25 (s, 2H, CH₂), 4.75 (d, 1H, J=9.2 Hz, CH—O), 4.89 (s, 2H, CH₂), 6.82-6.95 (m, 3H, ArH). ¹³C-NMR (CD₃OD, δ): 24.9, 26.7, 47.3, 47.6, 59.5, 65.7, 73.6, 116.4, 118.3, 120.3, 133.7, 145.1, 145.6, 166.4.

### DL-threo-2-Amino-1-(2,3-dihydrobenzo[b][1,4]dioxin-6-yl)-3-(pyrrolidin-1-yl)propan-1-ol Compound 6

To a stirred suspension of trans-(4,5-dihydro-5-(4-methoxyphenyl)oxazol-4-yl)(pyrrolidin-1-yl)methanone SLA 07080 (1.79 g, 5.44 mmol) in THF (220 mL) was slowly added at 0° C., in two portions, LiAlH₄ (1.28 g, 33.7 mmol). The mixture was stirred at RT for 3.5 h and quenched by a slow addition of water at 0° C. (350 mL). The white suspension was concentrated to remove THF and taken back in a mixture of CH₂Cl₂ (300 mL) and 1 N aqueous HCl (50 mL). The aqueous layer was basified to pH=10-11 by slow addition of 1 N aqueous NaOH. The organic layer was removed; two more extracts were combined and dried over MgSO₄, filtered and evaporated. Concentration afforded to a crude product as a yellow oil. This material was purified by column chromatography on silica (CH₂Cl₂:MeOH:NH₄OH 20%=94:5:1) to led to DL-threo-2-amino-1-(2,3-dihydrobenzo[b][1,4]dioxin-6-yl)-3-(pyrrolidin-1-yl)propan-1-ol Compound 6 (0.705 g, 46.5% yield) as a near colorless gum.

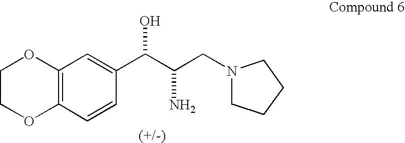

MW: 278.35; Yield: 46.5%; Colorless Gum. R_{f}: 0.20 (CH₂Cl₂:MeOH:NH₄OH 20%=94:5:1). ¹H-NMR (CDCl₃, δ): 1.70-1.85 (m, 4H, 2×CH₂), 2.40-2.70 (m, 6H, 3×CH₂N—), 3.05-3.15 (m, 1H, CH—N), 4.25 (s, 4H, CH₂O), 4.55 (d, 1H, J=2.2 Hz, CH—O), 5.30 (s, 1H, —OH), 6.75-6.90 (m, 3H, ArH).

### N-(DL-threo-1-(2,3-dihydrobenzo[b][1,4]dioxin-6-yl)-1-hydroxy-3-(pyrrolidin-1-yl)propan-2-yl)decanamide Compound 7

To a stirred solution of DL-threo-2-amino-1-(2,3-dihydrobenzo[b][1,4]dioxin-6-yl)-3-(pyrrolidin-1-yl)propan-1-ol BLE 04104 (0.186 g, 0.67 mmol) in 10 mL CH₂Cl₂ were added, in order, N-hydroxysuccinimide (0.081 g, 0.70 mmol) in 2 mL CH₂Cl₂, triethylamine (112 μL, 0.80 mmol) and decanoyl chloride (125 μL, 0.60 mmol). The mixture was stirred overnight at RT and then partitioned between CH₂Cl₂ and 1 N aqueous sodium hydroxide. The organic layer was dried over MgSO₄, filtered and evaporated and the residue obtained was purified by column chromatography on silica (CH₂Cl₂:MeOH=95:5). A white solid N-(DL-threo-1-(2,3-dihydrobenzo[b][1,4]dioxin-6-yl)-1-hydroxy-3-(pyrrolidin-1-yl)propan-2-yl)palmitamide Compound 7 was obtained (126 mg, 43.5% yield).

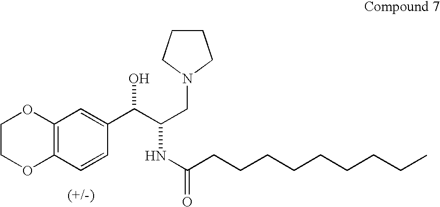

MW: 516.76; Yield: 43.5%; White Solid; Mp (° C.): 84.6. R_{f}: 0.40 (MeOH:CH₂Cl₂=10:90). ¹H-NMR (CDCl₃, δ): 0.88 (t, 3H, J=6.7 Hz, CH₃), 1.12-1.39 (m, 12H), 1.40-1.60 (m, 2H, CH₂), 1.72-1.90 (m, 4H, 2×CH₂), 2.10 (t, 2H, J=6.7 Hz, CH₂), 2.55-2.90 (m, 6H), 4.13-4.30 (m, 1H, CH—N), 4.24 (s, 4H, CH₂N), 4.91 (d, 1H, J=3.3 Hz, CH—O), 5.90 (d, 1H, J=7.4 Hz, NH), 6.75-6.88 (m, 3H, ArH), OH not seen. ¹³C-NMR (CDCl₃, δ): 14.1, 22.7, 23.6 (2×C), 25.6, 29.1, 29.3, 31.9, 36.8, 52.3, 55.1 (2×C), 57.7, 64.3 (2×C), 75.2, 77.2, 115.0, 117.0, 118.9, 134.4, 142.8, 143.4, 173.5, 174.8. MS-ESI m/z (% rel. Int.): 433.1 ([MH]⁺, 100). HPLC: Method A, detection UV 280 nm, Compound 7, RT=5.2 min, peak area 96.2%.

### N-(DL-threo-1-(2,3-Dihydrobenzo[b][1,4]dioxin-6-O-1-hydroxy-3-(pyrrolidin-1-yl)propan-2-yl)palmitamide Compound 8

To a stirred solution of DL-threo-2-amino-1-(2,3-dihydrobenzo[b][1,4]dioxin-6-yl)-3-(pyrrolidin-1-yl)propan-1-ol BLE 04104 (0.158 g, 0.57 mmol) in 10 mL CH₂Cl₂ were added, in order, N-hydroxysuccinimide (0.068 g, 0.59 mmol) in 2 ml CH₂Cl₂, triethylamine (95 μL, 0.68 mmol) and palmitoyl chloride (155 μL, 0.511 mmol) in 3 mL CH₂Cl₂. The mixture was stirred overnight at RT and then partitioned between CH₂Cl₂ and 1 N aqueous sodium hydroxyde. The organic layer was purified by column chromatography on silica using as eluent CH₂Cl₂:MeOH=95:5. A white solid N-(DL-threo-1-(2,3-dihydrobenzo[b][1,4]dioxin-6-yl)-1-hydroxy-3-(pyrrolidin-1-yl)propan-2-yl)palmitamide Compound 8 was obtained (148 mg, 50.4% yield).

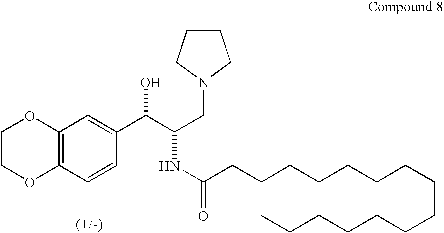

MW: 516.7; Yield: 50.4%; White Solid; Mp (° C.): 66.4. R_{f}: 0.50 (MeOH:CH₂Cl₂=10:90). ¹H-NMR (CDCl₃, δ): 0.88 (t, 3H, J=6.7 Hz, CH₃), 1.15-1.35 (m, 24H), 1.45-1.58 (m, 2H, CH₂), 1.75-1.90 (m, 4H, 2×CH₂), 2.10 (t, 2H, J=7.4 Hz, CH₂), 2.61 (s, 1H, OH), 2.52-2.72 (m, 4H), 2.72-2.92 (m, 2H), 4.15-4.22 (m, 1H, CH—N), 4.24 (s, 4H, CH₂N), 4.92 (d, 1H, J=3.3 Hz, CH—O), 6.08 (d, 1H, J=7.4 Hz, NH), 6.75-6.90 (m, 3H, ArH). MS-ESI m/z (% rel. Int.): 517.2 ([MH]⁺, 100). HPLC: Method A, detection UV 280 nm, Compound 8 RT=6.60 min, peak area 97.2%.

### Preparation of DL-threo-2-Amino-1-(pyridin-4-yl)-3-(pyrrolidin-1-yl)propan-1-ol Compound 46

### Trans-(4,5-dihydro-5-(pyridin-3-yl)oxazol-4-yl)(pyrrolidin-1-yl)methanone BLE 04110B

A general method D for oxazolines formation is illustrated by the preparation of BLE 04110B: To a stirred and cooled (0° C.) solution of potassium hydroxide (0.55 g, 9.80 mmol) in methanol (10 mL) were added a mixture of 3-pyridine carboxaldehyde (1.03 mL, 10.84 mmol) and 2-isocyano-1-(pyrrolidin-1-yl)ethanone BLE 04098 (1.50 g, 10.86 mmol). The solution was stirred 3 h at 0° C. and then concentrated. The residue was partitioned between ethyl acetate (100 mL) and water. The organic layer was combined with two additional ethyl acetate extracts (2×100 mL), washed with aqueous sodium chloride and dried over MgSO₄, filtered and evaporated. Concentration afforded a crude product which was purified by column chromatography on silica (CH₂Cl₂:MeOH=98:2) to yield to trans-(4,5-dihydro-5-(pyridin-3-yl)oxazol-4-yl)(pyrrolidin-1-yl)methanone BLE 04110B (0.95 g, 39%) as a pale yellow pale solid.

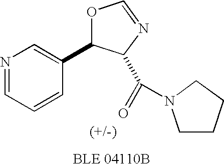

MW: 245.28; Yield: 39%; Yellow Pale Solid; Mp (° C.): 107.0. ¹H-NMR (CDCl₃, δ): 1.78-2.10 (m, 4H, 2×CH₂), 3.40-3.61 (m, 3H, CH₂N), 3.90-4.04 (m, 1H, CH₂N), 4.59 (dd, 1H, J=7.7 Hz, J=2.2 Hz, CH—N), 6.21 (d, 1H, J=7.7 Hz, CH—O), 7.04 (d, 1H, J=2.2 Hz, O—CH═N), 7.33 (m, 1H, ArH), 7.64 (m, 1H, ArH), 8.59 (d, 2H, J=2.8 Hz, ArH). ¹³C-NMR (CDCl₃, δ): 24.2, 26.0, 46.4, 46.6, 75.7, 79.3, 123.7, 133.5, 135.3, 147.6, 149.9, 155.2, 166.2.

### trans-(4,5-Dihydro-5-(pyridin-4-yl)oxazol-4-yl)(pyrrolidin-1-yl)methanone Compound 19

Compound 19 was prepared in accordance with method D using pyridine-4-carbaldehyde (1.88 mL, 19.76 mmol), KOH (1.01 g, 18.00 mmol) in methanol (18 mL) and 2-isocyano-1-(pyrrolidin-1-yl)ethanone BLE 04098 (2.73 g, 19.76 mmol). The residue was partitioned between ethyl acetate (200 mL) and water (150 mL). The organic layer was combined with additional ethyl acetate extracts (2×150 mL), washed with aqueous sodium chloride (2×150 mL) and dried over MgSO₄, filtered and evaporated. Trans-(4,5-dihydro-5-(pyridin-4-yl)oxazol-4-yl)(pyrrolidin-1-yl)methanone Compound 19 was obtained as a white solid (4.32 g, 98% yield).

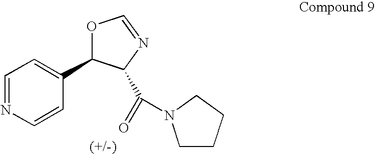

MW: 245.28; Yield: 98%; White Solid; Mp (° C.)=69.2. R_{f}: 0.65 (MeOH:CH₂Cl₂=10:90). ¹H-NMR (CDCl₃, δ): 1.78-2.06 (m, 4H, 2×CH₂), 3.44-3.60 (m, 3H, CH₂N), 3.90-4.01 (m, 1H, CH₂N), 4.52 (dd, 1H, J=7.9 Hz, J=2.2 Hz, CH—N), 6.19 (d, J=7.9 Hz, 1H, CH—O), 7.03 (d, 1H, J=2.2 Hz, N═CH—O), 7.24 (dd, 2H, J=4.5 Hz, J=1.5 Hz, ArH), 8.61 (dd, 2H, J=4.5 Hz, J=1.5 Hz, ArH).

A general method for the acidic hydrolysis of oxazolines (Method E) is illustrated in the preparation of Compound 20 which is a substituted propionic acid amide and is made from the oxazoline intermediate BLE 04110B which can be prepared in accordance with General Synthetic Scheme 1.

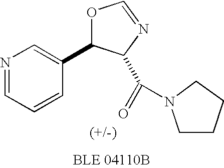

### DL-threo-2-Amino-3-hydroxy-3-(pyridin-3-yl)-1-(pyrrolidin-1-yl)propan-1-one dihydrochloride Compound 20

To a solution of trans-(4,5-dihydro-5-(pyridin-3-yl)oxazol-4-yl)(pyrrolidin-1-yl)-methanone BLE 04110B (0.932 g, 3.80 mmol) in methanol (10 mL) was added hydrochloric acid 37% (1.2 mL). After heating (50° C.) the mixture for 2.25 h the reaction mixture was concentrated and the crude product was coevaporated twice with ethyl acetate. After trituration with ethyl acetate, filtration and drying DL-threo-2-amino-3-hydroxy-3-(pyridin-3-yl)-1-(pyrrolidin-1-yl)propan-1-one dihydrochloride Compound 20 was obtained as a white solid (1.10 g, 94% yield).

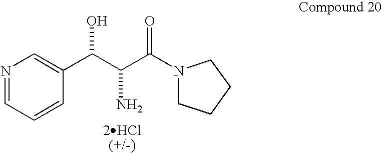

MW: 308.2; Yield: 94%; White Solid; Mp (° C.): 123.4. ¹H-NMR (CD₃OD, δ): 1.65-2.00 (m, 4H, 2×CH₂), 2.82-3.11 (m, 1H, —CH₂N), 3.30-3.57 (m, 2H, CH₂N), 3.57-3.77 (m, 1H, CH₂N), 4.54 (d, 1H, J=5.3 Hz, CH—N), 5.38 (d, 1H, J=5.3 Hz, CH—O), 8.15 (dd, 1H, J=7.6 Hz, J=5.0 Hz, ArH), 8.68 (d, 1H, J=7.6 Hz, ArH), 8.89 (d, 1H, J=7.6 Hz, ArH), 8.96 (s, 1H, ArH). ¹³C-NMR (CD₃OD): 24.9, 26.9, 47.7, 48.2, 58.1, 69.6, 128.7, 141.5, 141.6, 143.1, 146.5, 165.4.

### DL-threo-2-Amino-3-hydroxy-3-(pyridin-4-yl)-1-(pyrrolidin-1-yl)propan-1-one dihydrochloride Compound 22

Compound 22 was prepared following method E with trans-(4,5-dihydro-5-(pyridin-4-yl)oxazol-4-yl)(pyrrolidin-1-yl)methanone Compound 19 (0.750 g, 3.07 mmol), hydrochloric acid 37% (1.0 mL) and methanol (10 mL). After 3.0 h at 50° C. and work-up DL-threo-2-amino-3-hydroxy-3-(pyridin-4-yl)-1-(pyrrolidin-1-yl)propan-1-one dihydrochloride Compound 22 was obtained as a white solid (0.935 g, 99%).

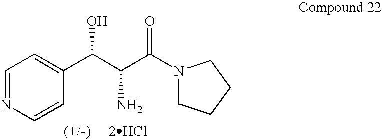

MW: 308.28; Yield: 99%; White Solid; Mp (° C.): 117.0. ¹H-NMR (CD₃OD, δ): 1.75-2.03 (m, 4H, 2×CH₂), 2.93-3.08 (m, 1H, CHN), 3.32-3.75 (m, 3H, 2×CH₂), 4.54 (d, 1H, J=5.9 Hz, CH N), 5.40 (d, 1H, J=5.9 Hz, CH—O), 8.21 (d, 2H, J=5.8 Hz, ArH), 8.94 (d, 2H, J=5.8 Hz, ArH). MS-ESI m/z (% rel. int.): 236.1 ([MH]⁺, 17), 219 (25), 148 (100). HPLC: Method A, detection UV 254 nm, Compound 22 RT=0.8 min, peak area 96.3%.

### DL-threo-2-Amino-1-(pyridin-4-yl)-3-(pyrrolidin-1-yl)propan-1-ol Compound 46

To a stirred suspension of DL-threo-2-amino-3-hydroxy-3-(pyridin-4-yl)-1-(pyrrolidin-1-yl)propan-1-one dihydrochloride Compound 22 (0.86 g, 2.80 mmol) in tetrahydrofuran (108 mL) under nitrogen atmosphere was slowly added, in two portions, lithium aluminium hydride (0.64 g, 16.82 mmol) at 0° C. The mixture reaction was stirred at RT for 20 h and quenched by a slow, dropwise addition of 2 N aqueous sodium hydroxyde (8.4 mL, 6 eq). The yellow precipitate was filtered. The organic layer was washed by water (80 mL) and the organic layer was removed and combined with additional ethyl acetate extracts (4×200 mL) and dried over MgSO₄, filtered and evaporated. The crude product was purified by column chromatography on silica (CH₂Cl₂:MeOH:NH₃=94:05:01). After evaporation and drying DL-threo-2-amino-1-(pyridin-4-yl)-3-(pyrrolidin-1-yl)propan-1-ol Compound 46 was obtained (0.075 g, 12%) as a pale yellow solid.

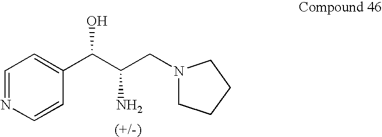

MW: 221.30; Yield: 12%; Pale Yellow Solid. R_{f}: 0.35 (CH₂Cl₂:MeOH:NH₃=90:08:02). ¹H-NMR (CD₃OD, δ): 1.60-1.80 (m, 4H, 2×CH₂), 2.30-2.80 (m, 6H, 3×CH₂N), 3.14-3.19 (m, 1H, CH—NH₂), 4.68 (d, 1H, J=3.0 Hz, CH—O), 7.30 (d, 2H, J=6.0 Hz, ArH), 8.55 (d, 2H, J=6.0 Hz, ArH). ¹³C-NMR (CD₃OD, δ): 23.5 (2×C), 54.1, 54.7 (2×C), 60.1, 74.5, 121.4 (2×C), 149.5 (2×C), 152.1. MS-ESI m/z (rel. int.): 222.1 ([MH]⁺, 100), 205.0 (80), 189.0 (45), 151.0 (70), 134.0 (42), 121.9 (100), 107.9 (40).

1. A method for treating a cognitive disorder, the method comprising administering to a patient in need of such treatment a compound of the following structure: 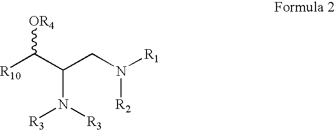
wherein R₁ is H or alkyl of 1 to 6 carbons,
R₂ is H, alkyl of 1 to 6 carbons or the R₁ and R₂ groups together with the nitrogen form a saturated or unsaturated 4, 5, 6 or 7 membered ring that optionally includes one or two heteroatoms independently selected from N, O and S, said 4, 5, 6 or 7 membered ring optionally being substituted with a halogen or with an alkyl group having 1 to 6 alkyl groups;
R₃ is independently selected from H, alkyl of 1 to 20 carbons, aryl or heteroaryl, aryl-alkyl or heteroaryl-alkyl wherein the alkyl moiety is has 1 to 4 carbons, cycloalkyl of 3 to 6 carbons, said aryl or heteroaryl groups being optionally substituted with 1 to 3 groups independently selected from the group consisting of halogen, alkyl of 1 to 6 carbons, alkoxy of 1 to 6 carbons and thioxy of 1 to 6 carbons, or
R₃ is CO—R₇ or CO—O—R₇ wherein R₇ is H, alkyl of 1 to 1 to 20 carbons, benzyl, alkyl of 1 to 20 carbons substituted with and NH₂ group, with a NHCOOalkyl or with an NH—COalkyl group wherein the alkyl group has one to 6 carbons, or R₇ is aryl, heteroaryl, aryl-alkyl or heteroaryl-alkyl wherein the alkyl moiety is branched or unbranched and has 1 to 4 carbons, said aryl or heteroaryl groups being optionally substituted with 1 to 3 groups independently selected from the group consisting of halogen, alkyl of 1 to 6 carbons, alkoxy of 1 to 6 carbons and thioxy of 1 to 6 carbons;
R₄ is H, alkyl of 1 to 6 carbons or CO—R₈ wherein R₈ is alkyl of 1 to 6 carbons;
the wavy lines represent bonds connected to carbons having R or S configuration, and
R₁₀ is selected from the groups of formulas (i) and (ii) 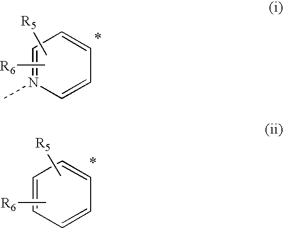
wherein the * indicates the carbon atom to which the remaining moiety of the molecule is attached;
R₅ and R₆ independently are H, alkyl of 1 to 6 carbons, halogen, alkoxy of 1 to 6 carbons or the R₅ and R₆ groups together with the atoms to which they are attached jointly form a carbocyclic or a heterocyclic ring, the carbocyclic ring having 5 or 6 atoms in the ring, the heterocyclic ring having 5 or 6 atoms in the ring and 1 to 3 heteroatoms independently selected from N, O and S, and said carbocyclic or heterocyclic ring jointly formed by R₅ and R₆ being optionally substituted with 1 to 6 R₉ groups wherein R₉ is independently selected from halogen, alkyl of 1 to 6 carbons, alkoxy of 1 to 6 carbons, or a pharmaceutically acceptable salt of said compound;
with the proviso:
that when R₁₀ has formula (II) then the claim does not include compounds wherein R₄ is hydrogen and R₁ and R₂ jointly with the nitrogen form a morpholin or a pyrrolidin ring and wherein R₅ and R₆ both are H or one of R₅ and R₆ is OCH₃ and the other is H.

2. The method of claim 1, wherein R₁₀ represents the formula (i).

3. The method of claim 1, wherein R₁₀ represents the formula (ii).

4. The method of claim 2, wherein the compound has the formula 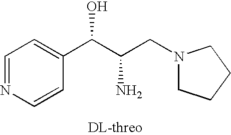
wherein R₅ and R₆ are independently selected from H, alkyl and alkoxy and R₄ is H or CO—R₈ or a pharmaceutically acceptable salt of said compound.

5. The method of claim 4, wherein the compound has the formula 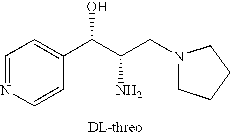
or a pharmaceutically acceptable salt of said compound.

6. The method of claim 3, wherein the compound has the formula 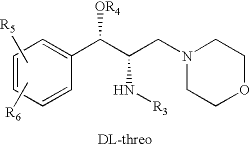
wherein R₃ is CO—R₇ or CO—O—R₇, R₄ is CO—R₈ and R₅ and R₆ are independently selected from H, alkyl of 1 to 6 carbons and alkoxy of 1 to 6 carbons or any other pharmaceutically acceptable salt of said compound.

7. The method of claim 6, wherein the compound has the formula 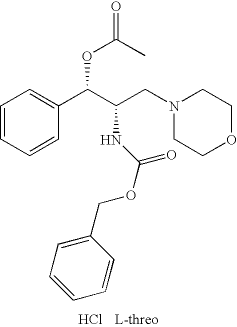
or any other pharmaceutically acceptable salt of said compound.

8. A method for treating a cognitive disorder, the method comprising administering to a patient in need of such treatment a compound of the following structure: 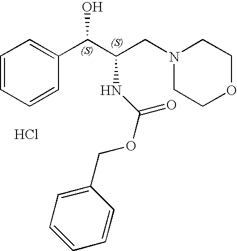
or any other pharmaceutically acceptable salt of said compound.

9. A method for treating a cognitive disorder, the method comprising administering to a patient in need of such treatment a compound of the following structure 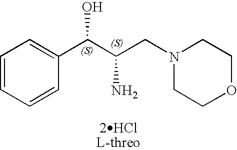
or any other pharmaceutically acceptable salt of said compound.

10. The method of claim 1, wherein the cognitive disorder is selected from the group consisting of an agnosia, an amnesia, an aphasia, an apraxia, a delirium, a dementia, and a learning disorder.

11. The method of claim 10, wherein the cognitive disorder is selected from the group consisting of AIDS dementia complex, Binswanger's disease, dementia with Lewy Bodies, frontotemporal dementia, mild cognitive impairment, multi-infarct dementia, Pick's disease, semantic dementia, senile dementia, and vascular dementia.

12. The method of claim 10, wherein the learning disorder is selected from the group consisting of Asperger's syndrome, attention deficit disorder,
attention deficit hyperactivity disorder, autism, childhood disintegrative disorder, and Rett syndrome.

13. The method of claim 10, wherein the aphasia is progressive non-fluent aphasia.

14. The method of claim 1, wherein the cognitive disorder is associated with neurodegenerative disease, injury to the brain, psychiatric disorders, or chronic pain.

15. The method of claim 14, wherein the neurodegenerative disease is selected from the group consisting of Alzheimer's disease, corticobasal degeneration, Creutzfeldt-Jacob disease, frontotemporal lobar degeneration, Huntington disease, multiple sclerosis, normal pressure hydrocephalus, organic chronic brain syndrome, Parkinson's disease, Pick disease, progressive supranuclear palsy, and senile dementia (Alzheimer type).

16. The method of claim 14, wherein the injury to the brain is selected from the group consisting of chronic subdural hematoma, concussion, intracerebral hemorrhage, encephalitis, meningitis, septicemia, drug intoxication, and drug abuse.

17. The method of claim 14, wherein the psychiatric disorders are selected from the group consisting of anxiety disorders, dissociative disorders, mood disorders, schizophrenia, and somatoform and factitious disorders.
